(19) United States
(12) Patent Application Publication
(10) Pub. No.: US 2023/0017738 A1
(43) Pub. Date: Jan. 19, 2023

(54) METHOD, APPARATUS AND SYSTEM FOR CONTROLLING AN IMAGE CAPTURE DEVICE DURING SURGERY

(71) Applicant: Sony Group Corporation, Tokyo (JP)

(72) Inventors: Bernadette ELLIOTT-BOWMAN, London (GB); Christopher WRIGHT, London (GB); Akinori KAMODA, Tokyo (JP); Yohei KURODA, Tokyo (JP)

(73) Assignee: Sony Group Corporation, Tokyo (JP)

(21) Appl. No.: 17/784,107

(22) PCT Filed: Nov. 5, 2020
(86) PCT No.: PCT/JP2020/041392

§ 371 Date: Jun. 10, 2022

(30) Foreign Application Priority Data
Dec. 19, 2019 (EP) 19218109.7

(51) Int. Cl. A61B 1/045; A61B 1/00

(52) U.S. Cl. CPC A61B 1/045; A61B 1/00149; A61B 1/00039; A61B 1/00045

(57) ABSTRACT

A system for controlling a medical image capture device during surgery, the system including: circuitry configured to receive a first image of the surgical scene, captured by the medical image capture device from a first viewpoint, and additional information of the scene; determine, for the medical image capture device, in accordance with the additional information and previous viewpoint information of surgical scenes, one or more candidate viewpoints from which to obtain an image of the surgical scene; provide, in accordance with the first image of the surgical scene, for each of the one or more candidate viewpoints, a simulated image of the surgical scene from the candidate viewpoint; control the medical image capture device to obtain an image of the surgical scene from the candidate viewpoint corresponding to a selection of one of the one or more simulated images of the surgical scene.

### FIELD

The present disclosure relates to a method, apparatus and system for controlling an image capture device during surgery.

### BACKGROUND

The “background” description provided herein is for the purpose of generally presenting the context of the disclosure. Work of the presently named inventors, to the extent it is described in the background section, as well as aspects of the description which may not otherwise qualify as prior art at the time of filing, are neither expressly or impliedly admitted as prior art against the present invention.

In recent years, significant technological developments in medical systems and equipment have been achieved. Computer assisted surgical systems, such as robotic surgical systems, now often work alongside a human surgeon during surgery. These computer assisted surgery systems include master-slave type robotic systems in which a human surgeon operates a master apparatus in order to control the operations of slave device during surgery.

Computer assisted camera systems, such as robotic camera systems, are used in a surgical environment to provide critical visual information to a human operator or surgeon. These computer assisted camera systems may be equipped with a single camera capturing and providing a view of surgical action within the scene. Alternatively, these computer assisted camera systems may include a plurality of cameras which each capture a given view of the surgical action within the scene.

In certain circumstances, it may be necessary to reposition a medical image capture apparatus supported by an articulated arm (e.g. through movement of the articulated arm) during surgery. This may be required if the view of the surgical scene provided by the computer assisted camera system becomes obstructed. Alternatively, this may be required as the surgeon progresses through the surgical procedure, as there may be differing requirements for the view from the computer assisted camera system of the surgical scene for each of the different surgical stages.

However, surgical scenes are inherently complex involving multiple independently moving components. Unnecessary repositioning of the camera system may delay the operation and cause unnecessary risk for the patient.

Furthermore, a reluctance to reposition the medical image capture apparatus may result in certain suboptimal viewpoints being tolerated by the surgeon during a surgical procedure. This may particularly be the case where an improved camera position cannot be readily identified by the surgeon. It is an aim of the present disclosure to address these issues.

### SUMMARY

According to a first aspect of the present disclosure, a system for controlling a medical image capture device during surgery is provided, the system including: circuitry configured to receive a first image of the surgical scene, captured by the medical image capture device from a first viewpoint, and additional information of the scene; determine, for the medical image capture device, in accordance with the additional information and previous viewpoint information of surgical scenes, one or more candidate viewpoints from which to obtain an image of the surgical scene; provide, in accordance with the first image of the surgical scene, for each of the one or more candidate viewpoints, a simulated image of the surgical scene from the candidate viewpoint; and control the medical image capture device to obtain an image of the surgical scene from the candidate viewpoint corresponding to a selection of one of the one or more simulated images of the surgical scene.

According to a second aspect of the present disclosure, a method of controlling a medical image capture device during surgery, the method comprising: receiving a first image of the surgical scene, captured by the medical image capture device from a first viewpoint, and additional information of the scene; determining, for the medical image capture device, in accordance with the additional information and previous viewpoint information of surgical scenes, one or more candidate viewpoints from which to obtain an image of the surgical scene; providing, in accordance with the first image of the surgical scene, for each of the one or more candidate viewpoints, a simulated image of the surgical scene from the candidate viewpoint; and controlling the medical image capture device to obtain an image of the surgical scene from the candidate viewpoint corresponding to a selection of one of the one or more simulated images of the surgical scene.

According to a third aspect of the present disclosure, a computer program product including instructions which, when the program is executed by a computer, cause the computer to carry out a method of controlling a medical image capture device during surgery, the method comprising: receiving a first image of the surgical scene, captured by the medical image capture device from a first viewpoint, and additional information of the scene; determining, for the medical image capture device, in accordance with the additional information and previous viewpoint information of surgical scenes, one or more candidate viewpoints from which to obtain an image of the surgical scene; providing, in accordance with the first image of the surgical scene, for each of the one or more candidate viewpoints, a simulated image of the surgical scene from the candidate viewpoint; and controlling the medical image capture device to obtain an image of the surgical scene from the candidate viewpoint corresponding to a selection of one of the one or more simulated images of the surgical scene.

According to embodiments of the disclosure, the apparatus for controlling an image capture device during surgery enables the surgeon to consider alternative viewpoints for a computer assisted camera system during surgery without having to repeatedly reposition the camera, thus enabling optimisation of computer assisted camera system viewpoint strategy without causing unnecessary delay to the surgical procedure. The present disclosure is not particularly limited to these advantageous effects, there may be others as would become apparent to the skilled person when reading the present disclosure.

The foregoing paragraphs have been provided by way of general introduction, and are not intended to limit the scope of the following claims. The described embodiments, together with further advantages, will be best understood by reference to the following detailed description taken in conjunction with the accompanying drawings.

### BRIEF DESCRIPTION OF DRAWINGS

A more complete appreciation of the disclosure and many of the attendant advantages thereof will be readily obtained as the same becomes better understood by reference to the following detailed description when considered in connection with the accompanying drawings.

FIG. 1 is a diagram illustrating an example of a schematic configuration of an endoscopic surgery system to which a medical support arm device according to the present disclosure can be applied.

FIG. 2 is a block diagram illustrating an example of functional configurations of a camera head and a CCU illustrated in FIG. 1.

FIG. 3 is an explanatory diagram illustrating a use example master apparatus according to the present disclosure.

FIG. 4 illustrates an example surgical situation to which embodiments of the present disclosure may be applied.

FIG. 5 illustrates an example of the image captured by an image capture device from a first viewpoint in accordance with embodiments of the disclosure.

FIG. 6 illustrates an apparatus for controlling an image capture device during surgery in accordance with embodiments of the disclosure.

FIG. 7 illustrates an example lookup table which can be used to determine candidate viewpoints in accordance with embodiments of the disclosure.

FIG. 8 shows an example illustration of the simulated images for the candidate viewpoint in accordance with embodiments of the disclosure.

FIG. 9 shows an example illustration of a user interface in accordance with embodiments of the disclosure.

FIG. 10 shows an example illustration of an image captured by an image capture device following a selection of a candidate viewpoint in accordance with embodiments of the disclosure.

FIG. 11 illustrates an apparatus for controlling an image capture device during surgery according to embodiments of the disclosure.

FIG. 12 shows an example illustration of a user interface in accordance with embodiments of the disclosure.

FIG. 13 shows an example setup of a computer assisted surgical system in accordance with embodiments of the present disclosure.

FIG. 14 illustrates a method of controlling an image capture device during surgery in accordance with embodiments of the disclosure.

FIG. 15 illustrates a computing device for controlling an image capture device during surgery in accordance with embodiments of the disclosure.

FIG. 16 schematically shows a first example of a computer assisted surgery system to which the present technique is applicable.

FIG. 17 schematically shows a second example of a computer assisted surgery system to which the present technique is applicable.

FIG. 18 schematically shows a third example of a computer assisted surgery system to which the present technique is applicable.

FIG. 19 schematically shows a fourth example of a computer assisted surgery system to which the present technique is applicable.

FIG. 20 schematically shows an example of an arm unit.

### DESCRIPTION OF EMBODIMENTS

Referring now to the drawings, wherein like reference numerals designate identical or corresponding parts throughout the several views.

### <<1. Basic Configuration>>

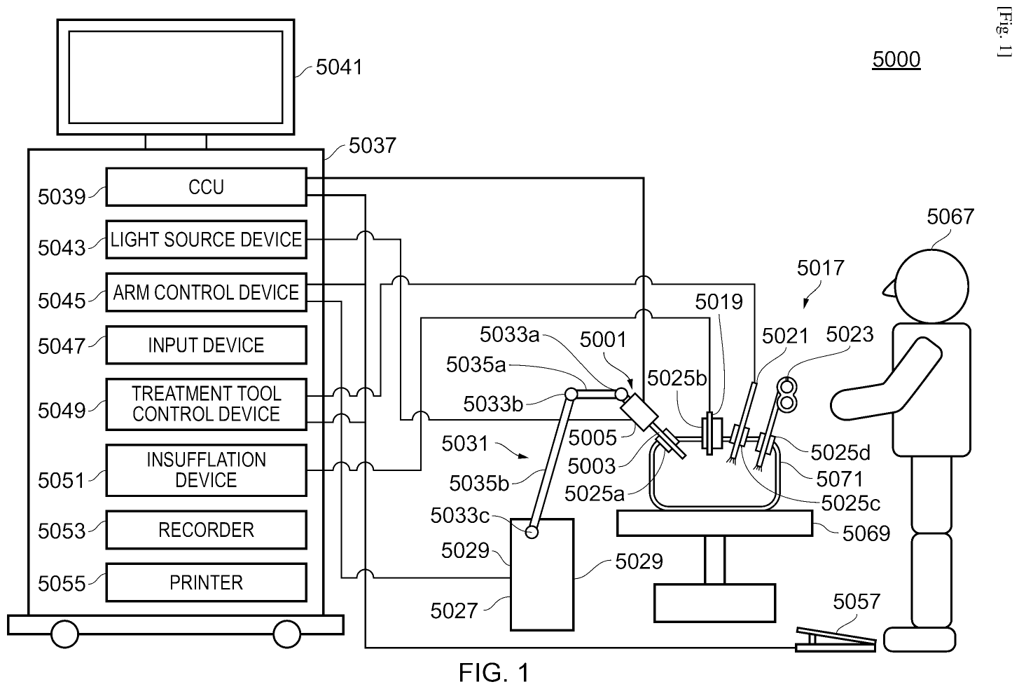

First, a basic configuration of an endoscopic surgery system to which embodiments of the disclosure may be applied will be described with reference to FIGS. 1 to 3 of the present disclosure.

### <1.1. Configuration Example of Endoscopic Surgery System>

FIG. 1 is a diagram illustrating an example of a schematic configuration of an endoscopic surgery system 5000 to which the technology according to the present disclosure can be applied. FIG. 1 illustrates a state where an operator (doctor) 5067 is conducting surgery to a patient 5071 on a patient bed 5069 using the endoscopic surgery system 5000. As illustrated, the endoscopic surgery system 5000 is constituted by an endoscope 5001, other surgical tools 5017, and a support arm device 5027 supporting the endoscope 5001, and a cart 5037 on which various devices for endoscopic surgery are mounted.

In the endoscopic surgery, the abdominal wall is punctured with a plurality of tubular hole-opening instruments called trocars 5025a to 5025d instead of cutting the abdominal wall to open the abdomen.

Then, a lens barrel 5003 of the endoscope 5001 and the other surgical tools 5017 are inserted into a body cavity of the patient 5071 through the trocars 5025a to 5025d. In the illustrated example, as the other surgical tools 5017, an insufflation tube 5019, an energy treatment tool 5021, and forceps 5023 are inserted into the body cavity of the patient 5071. Furthermore, the energy treatment tool 5021 is a treatment tool that performs incision and peeling of a tissue, sealing of a blood vessel, or the like using high-frequency current or ultrasonic vibration. However, the illustrated surgical tool 5017 is merely an example, and various surgical tools generally used in endoscopic surgery, for example, tweezers, a retractor, and the like may be used as the surgical tool 5017.

An image of an operation site in the body cavity of the patient 5071 captured by the endoscope 5001 is displayed on a display device 5041. The operator 5067 performs treatment, for example, to excise an affected site using the energy treatment tool 5021 or the forceps 5023 while viewing the image of the operation site displayed by the display device 5041 in real time. Note that the insufflation tube 5019, the energy treatment tool 5021, and the forceps 5023 are supported by the operator 5067, an assistant, or the like during surgery although not illustrated.

(Support Arm Device)

The support arm device 5027 includes an arm unit 5031 extending from a base unit 5029. In the illustrated example, the arm unit 5031 is a multi-joint arm constituted by joints 5033a, 5033b, and 5033c and links 5035a and 5035b, and is driven by control from an arm control device 5045. The arm unit 5031 has a distal end to which the endoscope 5001 can be connected. The endoscope 5001 is supported by the arm unit 5031, and a position and a posture thereof are controlled. With the configuration, it is possible to realize stable fixing of the position of the endoscope 5001.

(Endoscope)

The endoscope 5001 is constituted by the lens barrel 5003 having a region of a predetermined length from a distal end that is inserted into the body cavity of the patient 5071, and a camera head 5005 connected to a proximal end of the lens barrel 5003. Although the endoscope 5001 configured as a so-called rigid scope having the rigid lens barrel 5003 is illustrated in the illustrated example, the endoscope 5001 may be configured as a so-called flexible scope having the flexible lens barrel 5003.

An opening portion into which an objective lens is fitted is provided at the distal end of the lens barrel 5003. A light source device 5043 is connected to the endoscope 5001, and light generated by the light source device 5043 is guided to the distal end of the lens barrel by a light guide extended inside the lens barrel 5003 and is emitted toward an observation object in the body cavity of the patient 5071 through the objective lens. Note that the endoscope 5001 may be a forward-viewing scope, an oblique-viewing scope, or a side-viewing scope.

An optical system and an imaging element are provided inside the camera head 5005, and reflected light (observation light) from the observation object is collected on the imaging element by the optical system. The observation light is photoelectrically converted by the imaging element, and an electric signal corresponding to the observation light, in other words, an image signal corresponding to an observation image is generated. The image signal is transmitted as RAW data to a camera control unit (CCU) 5039.

Note that the camera head 5005 is equipped with a function of adjusting magnification and a focal length by properly driving the optical system.

Note that a plurality of imaging elements may be provided in the camera head 5005, for example, in order to cope with stereoscopic viewing (3D display) or the like. In this case, a plurality of relay optical systems is provided inside the lens barrel 5003 in order to guide the observation light to each of the plurality of imaging elements.

(Various Devices Equipped in Cart)

The CCU 5039 is configured using a central processing unit (CPU), a graphics processing unit (GPU), or the like, and integrally controls operations of the endoscope 5001 and the display device 5041.

Specifically, the CCU 5039 performs various types of image processing, for example, development processing (demosaicing processing) or the like on an image signal received from the camera head 5005 to display an image based on the image signal. The CCU 5039 provides the image signal subjected to the image processing to the display device 5041. Furthermore, the CCU 5039 transmits a control signal to the camera head 5005 and controls drive of the camera head 5005. The control signal may include information regarding imaging conditions such as magnification and a focal length.

The display device 5041 displays an image based on the image signal subjected to image processing by the CCU 5039 under the control of the CCU 5039. In a case where the endoscope 5001 is an endoscope compatible with high-resolution capturing, for example, 4K (the number of horizontal pixels of 3840×the number of vertical pixels of 2160), 8K (the number of horizontal pixels of 7680×the number of vertical pixels of 4320) or the like, and/or in a case of an endoscope compatible with 3D display, a device capable of high-resolution display and/or a device capable of 3D display can be used as the display device 5041 to be compatible with the above endoscopes, respectively. In the case of the endoscope compatible with the high-resolution capturing such as 4K and 8K, a more immersive feeling can be obtained by using the display device 5041 having a size of 55 inches or more. Furthermore, a plurality of the display devices 5041 having different resolutions and sizes may be provided in accordance with an application.

The light source device 5043 is configured using a light source such as a light emitting diode (LED), for example, and supplies irradiation light at the time of capturing an operation site to the endoscope 5001.

The arm control device 5045 is configured using a processor, for example, a CPU or the like, and operates according to a predetermined program to control the drive of the arm unit 5031 of the support arm device 5027 according to a predetermined control method.

The input device 5047 is an input interface with respect to the endoscopic surgery system 5000. A user can input various types of information and instructions to the endoscopic surgery system 5000 via the input device 5047. For example, the user inputs various types of information regarding surgery, such as information regarding a patient's body and information regarding surgical operation technology via the input device 5047. Furthermore, for example, the user inputs an instruction to drive the arm unit 5031, an instruction to change an imaging condition (a type of irradiated light, magnification, a focal length, or the like) using the endoscope 5001, an instruction to drive the energy treatment tool 5021, and the like via the input device 5047.

The type of the input device 5047 is not limited, and the input device 5047 may be various known input devices. For example, a mouse, a keyboard, a touch panel, a switch, a foot switch 5057 and/or a lever can be applied as the input device 5047. In a case where a touch panel is used as the input device 5047, the touch panel may be provided on a display surface of the display device 5041.

Alternatively, the input device 5047 is, for example, a device to be mounted by the user, such as a glasses-type wearable device and a head-mounted display (HMD), and various inputs are performed in accordance with a gesture or a line of sight of the user detected by these devices. Furthermore, the input device 5047 includes a camera capable of detecting user's motion, and various inputs are performed in accordance with a gesture or a line of sight of the user detected from an image captured by the camera.

Moreover, the input device 5047 includes a microphone capable of collecting user's voice, and various inputs are performed using the voice through the microphone. In this manner, the input device 5047 is configured to be capable of inputting various types of information in a non-contact manner, and particularly, the user (for example, the operator 5067) belonging to a clean area can operate equipment belonging to an unclean area in a non-contact manner. Furthermore, the user can operate the equipment without releasing his/her hand from the possessed surgical tool, and thus, the convenience of the user is improved.

The treatment tool control device 5049 controls the drive of the energy treatment tool 5021 for cauterization of a tissue, an incision, sealing of a blood vessel, or the like. An insufflation device 5051 sends a gas into a body cavity through the insufflation tube 5019 in order t to inflate the body cavity of the patient 5071 for the purpose of securing a visual field by the endoscope 5001 and securing a working space for the operator. A recorder 5053 is a device capable of recording various types of information regarding surgery. A printer 5055 is a device capable of printing various types of information regarding surgery in various formats such as text, an image, and a graph.

Hereinafter, a particularly characteristic configuration in the endoscopic surgery system 5000 will be described in more detail.

(Support Arm Device)

The support arm device 5027 includes the base unit 5029 as a base and the arm unit 5031 extending from the base unit 5029. Although the arm unit 5031 is constituted by the plurality of joints 5033a, 5033b, and 5033c, and the plurality of links 5035a and 5035b connected by the joint 5033b in the illustrated example,

FIG. 1 illustrates the configuration of the arm unit 5031 in a simplified manner for the sake of simplicity. Actually, each shape, the number, and the arrangement of the joints 5033a to 5033c and the links 5035a and 5035b, a direction of a rotation axis of each of the joints 5033a to 5033c, and the like are appropriately set such that the arm unit 5031 has a desired degree of freedom. For example, the arm unit 5031 can be preferably configured to have the degree of freedom equal to or greater than six degrees of freedom. With the configuration, the endoscope 5001 can be freely moved within a movable range of the arm unit 5031, and thus, it is possible to insert the lens barrel 5003 of the endoscope 5001 into the body cavity of the patient 5071 from a desired direction.

Actuators are provided in the joints 5033a to 5033c, and the joints 5033a to 5033c are configured to be rotatable about a predetermined rotation axis by the drive of the actuators. As the drive of the actuator is controlled by the arm control device 5045, each rotation angle of the joints 5033a to 5033c is controlled, and the drive of the arm unit 5031 is controlled. With the configuration, the control of the position and the posture of the endoscope 5001 can be realized. At this time, the arm control device 5045 can control the drive of the arm unit 5031 by various known control methods such as force control or position control.

For example, the position and posture of the endoscope 5001 may be controlled as the operator 5067 appropriately performs an operation input via the input device 5047 (including the foot switch 5057) and the drive of the arm unit 5031 is appropriately controlled by the arm control device 5045 according to the operation input. Through such control, the endoscope 5001 at the distal end of the arm unit 5031 can be moved from an arbitrary position to an arbitrary position, and then, fixedly supported at a position after the movement. Note that the arm unit 5031 may be operated in a so-called master-slave manner. In this case, the arm unit 5031 can be remotely operated by the user via the input device 5047 installed at a place distant from an operating room.

Furthermore, in a case where the force control is applied, the arm control device 5045 may receive an external force from the user and perform so-called power assist control to drive the actuators of the joints 5033a to 5033c such that the arm unit 5031 moves smoothly according to the external force. With the configuration, when the user moves the arm unit 5031 while directly touching the arm unit 5031, the arm unit 5031 can be moved with a relatively light force. Therefore, it is possible to more intuitively move the endoscope 5001 with a simpler operation, and it is possible to improve the convenience of the user.

Here, the endoscope 5001 has been generally supported by a doctor called a scopist in endoscopic surgery. In regard to this, it becomes possible to more reliably fix the position of the endoscope 5001 without human hands by using the support arm device 5027, and thus, it is possible to stably obtain an image of an operation site and to smoothly perform the surgery.

Note that the arm control device 5045 is not necessarily provided in the cart 5037.

Furthermore, the arm control device 5045 is not necessarily one device. For example, the arm control device 5045 may be provided at each of joints 5033a to 5033c of the arm unit 5031 of the support arm device 5027, or the drive control of the arm unit 5031 may be realized by the plurality of arm control devices 5045 cooperating with each other.

(Light Source Device)

The light source device 5043 supplies irradiation light at the time of capturing an operation site to the endoscope 5001. The light source device 5043 is configured using, for example, a white light source constituted by an LED, a laser light source, or a combination thereof. At this time, in a case where the white light source is constituted by a combination of RGB laser light sources, the output intensity and output timing of each color (each wavelength) can be controlled with high precision, and thus, it is possible to adjust white balance of a captured image in the light source device 5043. Furthermore, in this case, it is also possible to capture an image corresponding to each of RGB in a time-division manner by irradiating an observation object with laser light from each of the RGB laser light sources in a time-division manner and controlling the drive of the imaging element of the camera head 5005 in synchronization with an irradiation timing. According to this method, a color image can be obtained without providing a color filter in the imaging element.

Furthermore, the drive of the light source device 5043 may be controlled so as to change the intensity of light to be output every predetermined time. The drive of the imaging element of the camera head 5005 is controlled in synchronization with a timing of the change of the light intensity to acquire images in a time-division manner, and a so-called high dynamic range image without so-called crushed blacks and blown-out whites can be generated by combining the images.

Furthermore, the light source device 5043 may be configured to be capable of supplying light in a predetermined wavelength band which is compatible with special light observation. In the special light observation, for example, the wavelength dependency of light absorption in a body tissue is utilized, and light is emitted in a narrow band as compared to irradiation light during normal observation (in other words, white light), thereby performing so-called narrow band imaging (NBI) in which a predetermined tissue, such as a blood vessel in a superficial portion of a mucous membrane, is captured at a high contrast. Alternatively, fluorescent observation that obtains an image with fluorescent light generated by emitting excitation light may also be performed in the special light observation. In the fluorescence observation, it is possible to irradiate a body tissue with excitation light and observe fluorescent light from the body tissue (autofluorescence observation), to locally inject a reagent such as indocyanine green (ICG) into a body tissue and also irradiate the body tissue with excitation light corresponding to a fluorescence wavelength of the reagent to obtain a fluorescent image, or the like. The light source device 5043 can be configured to be capable of supplying narrow-band light and/or excitation light corresponding to such special light observation.

(Camera Head and CCU)

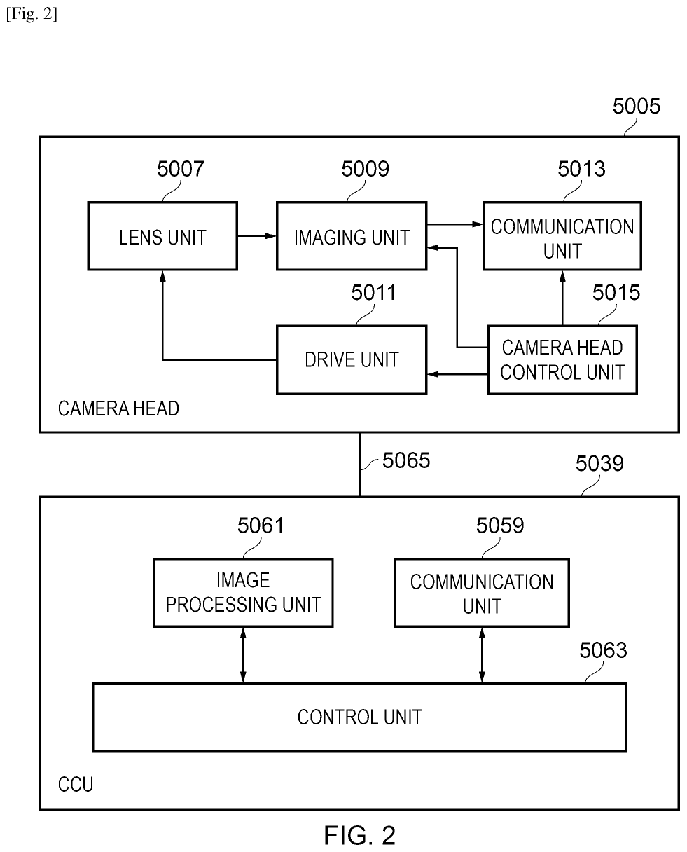

Functions of the camera head 5005 and the CCU 5039 of the endoscope 5001 will be described in more detail with reference to FIG. 2. FIG. 2 is a block diagram illustrating an example of functional configurations of the camera head 5005 and the CCU 5039 illustrated in FIG. 1.

The camera head 5005 has a lens unit 5007, an imaging unit 5009, a drive unit 5011, a communication unit 5013, and a camera head control unit 5015 as functions thereof with reference to FIG. 2. Furthermore, the CCU 5039 has a communication unit 5059, an image processing unit 5061, and a control unit 5063 as functions thereof. The camera head 5005 and the CCU 5039 are connected to be capable of bi-directional communication via a transmission cable 5065.

First, the functional configuration of the camera head 5005 will be described. The lens unit 5007 is an optical system provided at a connection portion with the lens barrel 5003. Observation light taken in from the distal end of the lens barrel 5003 is guided to the camera head 5005 and is incident onto the lens unit 5007. The lens unit 5007 is configured by combining a plurality of lenses including a zoom lens and a focus lens. Optical characteristics of the lens unit 5007 are adjusted such that observation light is collected on a light receiving surface of an imaging element of the imaging unit 5009. Furthermore, the zoom lens and the focus lens are configured such that positions on the optical axis thereof can be moved for adjustment of magnification and a focal length of a captured image.

The imaging unit 5009 is constituted by the imaging element, and is arranged at the subsequent stage of the lens unit 5007. The observation light having passed through the lens unit 5007 is collected on the light receiving surface of the imaging element, and an image signal corresponding to the observation image is generated by photoelectric conversion. The image signal generated by the imaging unit 5009 is provided to the communication unit 5013.

As the imaging element constituting the imaging unit 5009, for example, a complementary metal oxide semiconductor (CMOS) type image sensor that is capable of color capturing having the Bayer arrangement can be used. Note that, for example, an imaging element capable of being compatible with capturing of a high-resolution image of 4K or more may be used as the imaging element. Since the high-resolution image of an operation site can be obtained, the operator 5067 can grasp a situation of the operation site in more detail and can proceed surgery more smoothly.

Furthermore, the imaging element constituting the imaging unit 5009 is configured to have a pair of imaging elements to acquire image signals for a right eye and a left eye, respectively, compatible with 3D display. As the 3D display is performed, the operator 5067 can more accurately grasp a depth of a living tissue in the operation site. Note that a plurality of the lens units 5007 is provided to correspond to the respective imaging elements in a case where the imaging unit 5009 is configured in a multi-plate type.

Furthermore, the imaging unit 5009 is not necessarily provided in the camera head 5005. For example, the imaging unit 5009 may be provided inside the lens barrel 5003 just behind an objective lens.

The drive unit 5011 is configured using an actuator, and the zoom lens and the focus lens of the lens unit 5007 are moved along the optical axis by a predetermined distance under the control of the camera head control unit 5015. With the movement, the magnification and the focal length of the image captured by the imaging unit 5009 can be appropriately adjusted.

The communication unit 5013 is configured using a communication device to transmit and receive various types of information to and from the CCU 5039. The communication unit 5013 transmits an image signal obtained from the imaging unit 5009 as RAW data to the CCU 5039 via the transmission cable 5065. In this case, it is preferable that the image signal be transmitted by optical communication in order to display the captured image of the operation site with low latency. During surgery, the operator 5067 performs the surgery while observing a state of the affected site through the captured image, and thus, it is required to display a moving image of the operation site in real time as much as possible in order for a safer and more reliable surgery. In the case where the optical communication is performed, a photoelectric conversion module that converts an electric signal into an optical signal is provided in the communication unit 5013. The image signal is converted into the optical signal by the photoelectric conversion module, and then, is transmitted to the CCU 5039 via the transmission cable 5065.

Furthermore, the communication unit 5013 receives a control signal to control the drive of the camera head 5005 from the CCU 5039. The control signal includes information regarding imaging conditions such as information to designate a frame rate of a captured image, information to designate an exposure value at the time of imaging, and/or information to designate magnification and a focal length of a captured image, for example. The communication unit 5013 provides the received control signal to the camera head control unit 5015. Note that a control signal from the CCU 5039 may also be transmitted by optical communication. In this case, the communication unit 5013 is provided with a photoelectric conversion module that converts an optical signal into an electric signal, and the control signal is converted into the electrical signal by the photoelectric conversion module, and then, is provided to the camera head control unit 5015.

Note that the imaging conditions such as the above-described frame rate, exposure value, magnification, and focal length are automatically set by the control unit 5063 of the CCU 5039 on the basis of the acquired image signal. That is, the endoscope 5001 is equipped with so-called auto exposure (AE) function, auto focus (AF) function, and auto white balance (AWB) function.

The camera head control unit 5015 controls the drive of the camera head 5005 on the basis of the control signal from the CCU 5039 received via the communication unit 5013. For example, the camera head control unit 5015 controls the drive of the imaging element of the imaging unit 5009 on the basis of the information to designate the frame rate of the captured image and/or the information to designate the exposure at the time of imaging. Furthermore, for example, the camera head control unit 5015 appropriately moves the zoom lens and the focus lens of the lens unit 5007 via the drive unit 5011 on the basis of the information to designate the magnification and the focal length of the captured image.

Moreover, the camera head control unit 5015 may have a function of storing information to identify the lens barrel 5003 and the camera head 5005.

Note that the camera head 5005 can be made resistant to autoclave sterilization processing by arranging the configurations of the lens unit 5007, the imaging unit 5009, and the like in a sealed structure with high airtightness and waterproofness.

Next, the functional configuration of the CCU 5039 will be described. The communication unit 5059 is configured using a communication device to transmit and receive various types of information to and from the camera head 5005. The communication unit 5059 receives an image signal transmitted from the camera head 5005 via the transmission cable 5065. In this case, the image signal can be suitably transmitted by optical communication as described above. In this case, the communication unit 5059 is provided with a photoelectric conversion module that converts an optical signal into an electric signal to be compatible with the optical communication. The communication unit 5059 provides the image signal that has been converted into the electric signal to the image processing unit 5061.

Furthermore, the communication unit 5059 transmits a control signal to control the drive of the camera head 5005 to the camera head 5005. The control signal may also be transmitted by optical communication.

The image processing unit 5061 performs various types of image processing on the image signal which is RAW data transmitted from the camera head 5005. For examples, the image processing includes various types of known signal processing such as development processing, image quality improvement processing (band enhancement processing, super-resolution processing, noise reduction (NR) processing and/or camera shake correction processing, for example), and/or enlargement processing (electronic zoom processing). Furthermore, the image processing unit 5061 performs the detection processing on an image signal for performing AE, AF, and AWB.

The image processing unit 5061 is configured using a processor such as a CPU and a GPU, and the above-described image processing and detection processing can be performed when the processor operates according to a predetermined program. Note that, in a case where the image processing unit 5061 is constituted by a plurality of GPUs, the image processing unit 5061 appropriately divides information regarding the image signal and performs the image processing in parallel by the plurality of GPUs.

The control unit 5063 performs various types of control regarding imaging of an operation site using the endoscope 5001 and display of such a captured image. For example, the control unit 5063 generates a control signal to control the drive of the camera head 5005. At this time, in a case where an imaging condition is input by a user, the control unit 5063 generates the control signal on the basis of the input by the user. Alternatively, in a case where the endoscope 5001 is equipped with the AE function, the AF function, and the AWB function, the control unit 5063 appropriately calculates optimal exposure value, focal length, and white balance to generate the control signal in accordance with a result of the detection processing by the image processing unit 5061.

Furthermore, the control unit 5063 causes the display device 5041 to display the image of the operation site on the basis of the image signal subjected to the image processing by the image processing unit 5061.

At this time, the control unit 5063 recognizes various objects in the image of the operation site using various image recognition technologies. For example, the control unit 5063 detects a shape of an edge, a color, and the like of an object included in the operation site image, and thus, can recognize a surgical tool such as forceps, a specific living body part, bleeding, mist at the time of using the energy treatment tool 5021, and the like. When the display device 5041 is caused to display the image of the operation site, the control unit 5063 causes various types of surgical support information to be superimposed and displayed on the image of the operation site using such a recognition result. Since the surgical support information is superimposed and displayed, and presented to the operator 5067, it is possible to proceed the surgery more safely and reliably.

The transmission cable 5065 connecting the camera head 5005 and the CCU 5039 is an electric signal cable compatible with communication of an electric signal, an optical fiber compatible with optical communication, or a composite cable thereof.

Here, communication is performed in a wired manner using the transmission cable 5065 in the illustrated example, but the communication between the camera head 5005 and the CCU 5039 may be performed in a wireless manner. In the case where the communication between the two is performed in a wireless manner, it is not necessary to lay the transmission cable 5065 in the operating room, and thus, a situation in which movement of a medical staff is hindered by the transmission cable 5065 in the operating room can be resolved.

An example of the endoscopic surgery system 5000 to which the technology according to the present disclosure can be applied has been described as above. Note that the endoscopic surgery system 5000 has been described as an example here, but a system to which the technology according to the present disclosure can be applied is not limited to such an example. For example, the technology according to the present disclosure may be applied to a flexible endoscope system for inspection or a microscopic surgery system.

Alternatively, aspects of the present disclosure may be applied to a medical robot system including a master-slave medical robot system. In the medical robot system, a user (such as doctor 5067) operates a master apparatus (surgeon console) to transmit an operation command to a slave apparatus (bedside cart) through a wired or wireless communication means and remotely operate the slave apparatus. The medical robot system may also include a separate cart that contains some supporting hardware and software components, such as an electrosurgical unit (ESU), suction/irrigation pumps, and light source for the endoscope/microscope.

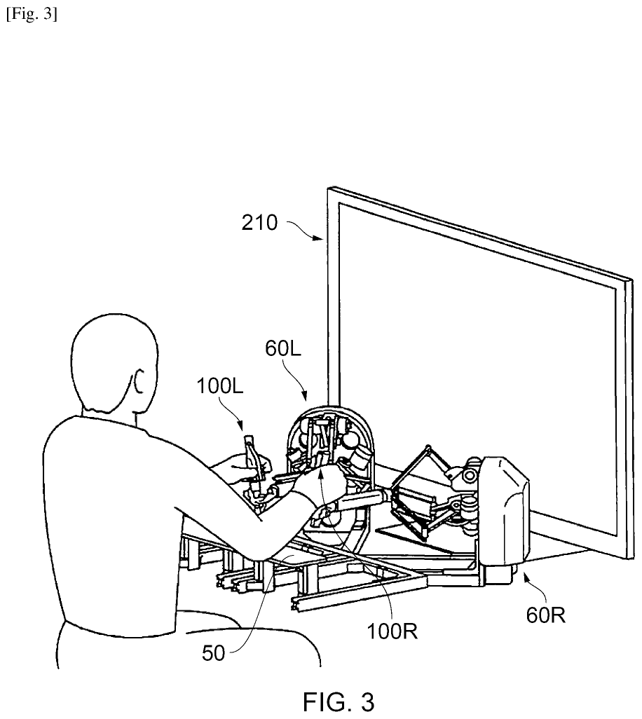

FIG. 3 illustrates a use example of the master apparatus 60 according to the present disclosure. In FIG. 3, two master apparatuses 60R and 60L for a right hand and a left hand are both provided. A surgeon puts both arms or both elbows on the supporting base 50, and uses the right hand and the left hand to grasp the operation portions 100R and 100L, respectively. In this state, the surgeon operates the operation portions 100R and 100L while watching a monitor 210 showing a surgical site. The surgeon may displace the positions or directions of the respective operation portions 100R and 100L to remotely operate the positions or directions of surgical instruments attached to slave apparatuses each of which is not illustrated, or use each surgical instrument to perform a grasping operation.

The basic configuration of example surgery systems applicable to embodiments of the disclosure has been described above with reference to FIGS. 1 to 3 of the present disclosure. Hereinafter, specific embodiments of the present disclosure will be described.

### <Controlling an Image Capture Device During Surgery>

As noted above, it is desirable that an apparatus is provided which enables optimisation of a viewpoint of a computer assisted camera system during surgery without disruption to the surgical procedure. As such, an apparatus, method and computer program product for controlling an image capture device during surgery is provided in accordance with embodiments of the disclosure.

The apparatus for controlling an image capture device during surgery will now be described with reference to an example surgical situation. However, it will be appreciated that the present disclosure is not particularly limited to this specific example, and may be applied to any such surgical situation as required.

### Example Situation

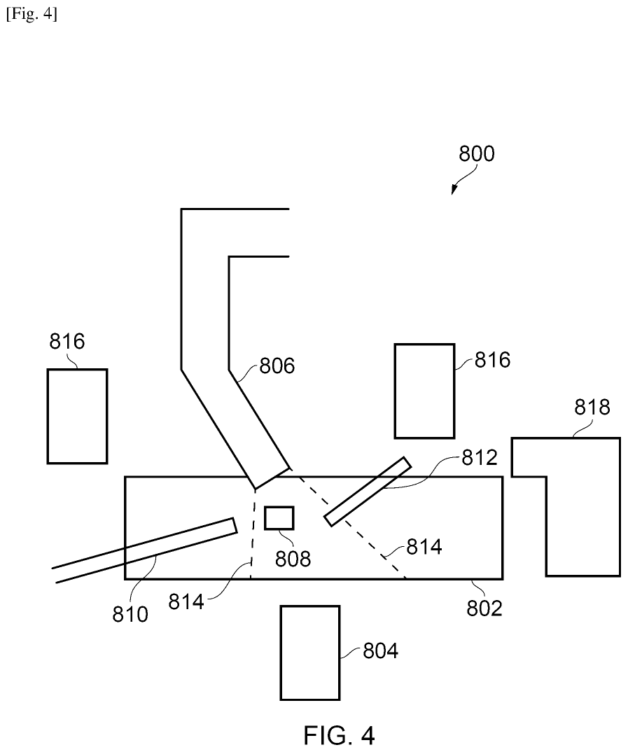

FIG. 4 illustrates an example surgical situation to which embodiments of the present disclosure may be applied.

In this example, a surgical scene 800 (such as an operating theatre) is shown. A patient 802 is being operated on by a surgeon 804. This may be a surgical procedure which requires the surgeon to perform an operation on a target region 808 of the patient. In this example, the surgery which the surgeon is performing is a laparoscopic surgery—however, the present application is not particularly limited in this regard. During the laparoscopic surgery, the surgeon is using one or more surgical tools and an endoscope (which is a scope attached to a camera head). These surgical tools and the endoscope are inserted into a patient's body cavity, through trocars (such as those described with reference to FIG. 1 of the present disclosure), in order to enable the surgeon to perform the laparoscopic surgery on the patient.

Now, in this example, the surgeon 804 is assisted during surgery by a computer assisted surgical system including a computer assisted camera system 806. The computer assisted surgical system may be a system such as those systems described with reference to FIGS. 1 to 3 of the present disclosure, for example.

In this example, the computer assisted camera system 806 includes a medical image capture device, such as an endoscope system including a scope and a camera head, which captures images of the scene 800 and provides the images to a display (not shown). The surgeon 804 can then view the images obtained by the computer assisted camera system 806 when performing the surgery on patient 802.

As noted above, during the surgical procedure, the surgeon 804 performs a treatment on a target region 808 of patient 802. In order to perform the treatment, the surgeon 804 may introduce one or more surgical tools 810 and 812 into the scene. In this specific example, surgical tool 810 may be a scalpel, while surgical tool 812 may be a suction device. Because the surgeon is operating on the target region 808, the computer assisted camera system is configured such that the image capture device of the computer assisted camera system captures images of the target region 808 of the patient 802. That is, the computer assisted camera system is configured such that the target region 808 falls within the field of view of the image capture device (the field of view of the image capture device being illustrated by the region encompassed by lines 814 in this example.

During surgery, the surgeon 804 is also assisted by one or more medical support staff and/or assistants 816. It is important that these medical support staff and/or assistants 816 are in close proximity to both the patient 802 and the surgeon 804 such that they can provide the necessary support and assistant to the surgeon 804 during the surgical procedure. For example, surgeon 804 may require that a medical assistant 816 passes the surgeon a particular tool or performs a particular task at a given stage during the surgical procedure.

Additional medical equipment 818 may also be located in the surgical scene. This equipment may include items such as an anaesthesia machine, instrument table, patient monitors, and the like. It is important that this equipment is provided in close proximity to the patient 802 and surgeon 808, such that the equipment can be readily accessed during the surgical procedure by the surgeon (or other surgical professionals within the surgical environment (such as a doctor who is responsible for the anaesthesia)) as required.

In some examples, such as endoscopic surgical procedures, the surgeon 808 may not be able to directly view the target region 808 of patient 802. That is, the computer assisted camera system 806 may provide the surgeon with the only available view of the target region. Moreover, even in situations whereby the surgeon can directly view the target region 808, the computer assisted camera system may provide an enhanced view of the target region 808 (such as a magnified view of the target region) upon which the surgeon depends in order to perform the surgery.

Accordingly, it is important that the computer assisted camera system provides the surgeon with a clear and/or unobstructed view of the target region. As such, substantial care may be taken in the initial configuration of the computer assisted camera system.

However, as the surgery progresses, dynamic elements within the surgical environment may impede the image which is obtained by the computer assisted camera system, resulting in a deterioration of the view of the scene provided to the surgeon 804. That is, the introduction of one or more additional surgical tools during the surgical procedure into the surgical environment may result in at least a partial obscuration of the target region from the viewpoint of the computer assisted camera system (that is, from the location at which the image capture device of the computer assisted camera system captures images of the target region 808).

Alternatively, the movements of the surgeon 804 and/or the support staff and assistants 816 may impede the ability of the image capture device of the computer assisted camera system to capture a clear image of the scene.

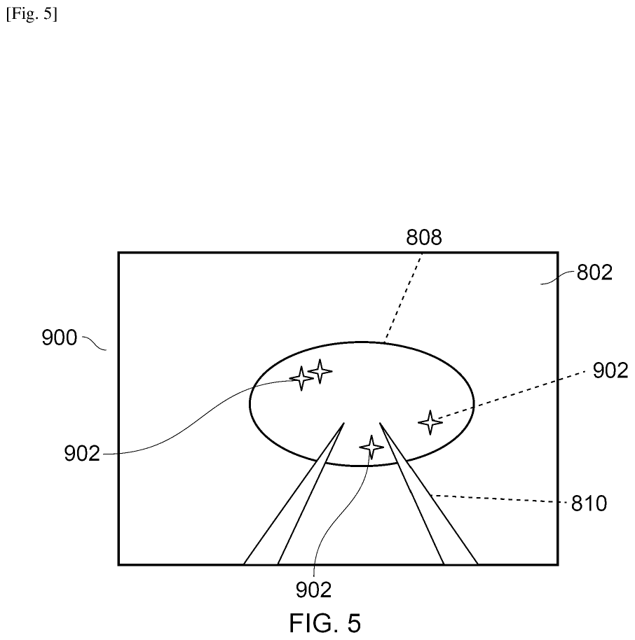

FIG. 5 illustrates an example of the image captured by an image capture device from a first viewpoint.

In FIG. 5, the image 900 of target region 808 of patient 802 captured by the image capture device of the computer assisted camera system 806 is shown. Surgical tool 810 is also seen in this image captured by the image capture device. Now, when the surgery began, the image capture device captured a clear image of the target region 808. However, at this time (that is, in at the time corresponding to the current image captured by the image capture device) the view of the scene captured by the image capture device has deteriorated.

Specifically, in this example, the surgeon can no longer obtain a clear view of the target region because significant glare and reflections 902 from the tissue surface of the target region have developed. These glare and reflection spots 902 may have developed due to changes in the target region and/or changes in the surgical environment, and prevent the surgeon from obtaining a clear view of the target region.

However, surgeon 804 may be unaware of whether there exist a more optimum position or viewpoint for the image capture device of the computer assisted camera system. Moreover, because of the delay to the surgical procedure which may be caused by a repositioning of the image capture device, the surgeon 804 is unwilling to try other viewpoints to see whether or not they reduce the glare and reflections.

Accordingly, an apparatus for controlling an image capture device during surgery is provided in accordance with embodiments of the disclosure.

Apparatus:

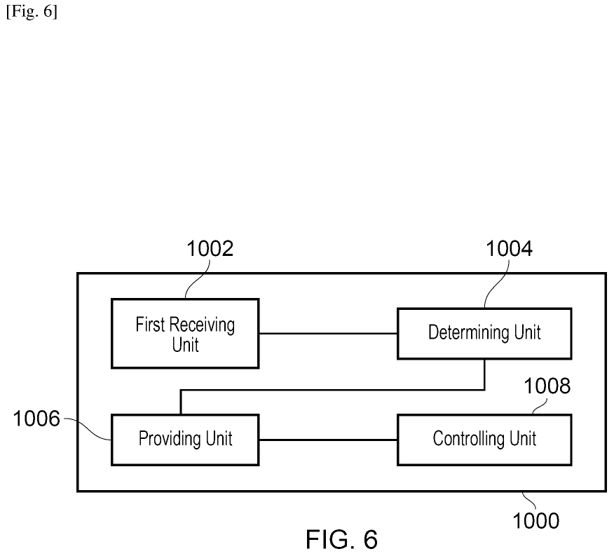

FIG. 6 illustrates an apparatus, or system, for controlling an image capture device (such as a medical image capture device) during surgery in accordance with embodiments of the disclosure.

The apparatus 1000 includes a first receiving unit 1002 configured to receive a first image of the surgical scene, captured by a medical image capture device from a first viewpoint, and additional information of the scene; a determining unit 1004, configured to determine, for the medical image capture device, in accordance with the additional information and previous viewpoint information of surgical scenes, one or more candidate viewpoints from which to obtain an image of the surgical scene; a providing unit 1006, configured to provide, in accordance with the first image of the surgical scene, for each of the one or more candidate viewpoints, a simulated image of the surgical scene from that candidate viewpoint; and a controlling unit 1008, configured to control the medical image capture device to obtain an image of the surgical scene from the candidate viewpoint corresponding to a selected one of the one or more simulated images of the surgical scene.

Returning to the example situation of FIG. 4 of the present disclosure, the apparatus 1000 may be connected to the arm control device (such as arm control device 5045 described with reference to FIG. 1) in order to control the movement of the image capture device. Alternatively, the apparatus 1000 may be connected to, or form part of a central processing unit. Features of the apparatus 1000 will now be described with reference to the example surgical situation of FIG. 4 of the present disclosure. However, it will be appreciated that the apparatus may be applied to any such surgical situation as required.

First Receiving Unit:

<First Image Data>

As described above, during surgery, the image capture device of the computer assisted camera system 1000 captures images of the surgical scene. The first receiving unit 1002 of apparatus 1000 is configured to receive the images captured by the image captured device as a first image (or image data). The first image thus provides the apparatus 1000 with information regarding the appearance of the surgical scene at the time the image was captured by the image capture device. In this example, the first image is therefore the same image that is displayed to a user (such as a surgeon) on a display device (such as displayed device 5041). That is, the first image shows the current appearance of the surgical scene. In this example the first image may therefore be image 900 as illustrated in FIG. 5 of the present disclosure.

It will be appreciated that the manner by which the receiving unit receives the first image data is not particularly limited. For example, the receiving unit can receive the image data from the image capture device by any suitable wired or wireless means. Moreover, the actual form of the image data will depend upon the type of image capture device which is used to capture the image data. In the present example, the image capture device may be an endoscopic device, a telescopic device a microscopic device or an exoscope device. As such, in this example, the image data acquired by the acquiring unit may be a high definition image, 4K image or 8K image of the scene, or the like. That is, any medical imagining device may be used in accordance with embodiments of the disclosure as required.

<Types of Additional Information>

In addition, the first receiving unit 1002 of the apparatus 1000 is further configured to receive additional information of the scene. Now, the form of this additional information is not particularly limited, and will vary in accordance with situation to which the embodiments of the disclosure are applied. Moreover, it will be appreciated that the apparatus 1000 may receive the additional information from a number of different sources depending on the type of the additional information which is being received. However, regardless of the form, it will be appreciated that the additional information is contextual information which provides the apparatus 1000 with a greater understanding of the surgical procedure being performed by the surgeon 804.

In certain examples, the additional information of the scene may include at least one of surgical and/or environmental information of the surgical scene.

In some examples, the environmental information may include information about the surgeon's working area. This may include information such as the location and orientation of the surgeon with respect to the target area of the patient, the working space around the surgeon, obstacles (such as surgical equipment) which are located within the area surrounding the surgeon; the lighting status (such as the lighting type and the lighting control information); orientation of the operating table with respect to the image capture device, or the like.

In some examples, the surgical information may include surgical tool information, providing the apparatus 1000 with a detailed understanding of the surgical tools used by the surgeon and their respective individual locations within the surgical scene. That is, in examples, the additional information may include surgical tool information such as: the type or types of tools which are located in the surgical scene; the locations of tools within the surgical scene; the usage status of the tools (whether a tool, such as an energy device, is activated, for example); information regarding how a tool is manipulated by the surgeon (such as whether a tool is held by the surgeon in both hands, or held by the supporting surgeon, for example); tool spatial and motion information (including velocity, trajectory, degree of tool activity (i.e. movements per minute) and end-effector separation between multiple tools); number of tool changes within a certain period of time; upcoming tools (such as which tool is being prepared by the assistant surgeon for use in a next stage of the surgical procedure, for example), or the like.

In some examples, the surgical information received may include information regarding the appearance of the surgical tissue and/or properties of the surgical tissue which will be operated on by the surgeon. For example, this may include information on the portion of the patient the surgeon will operate on (such as the heart or the lungs, for example), or the like.

In some examples, the surgical information may include procedural information related to the status of the surgery (such as the progress of the surgery), the specific type of surgery being performed by the surgeon (such as a standardised workflow for a given type of surgery). This information may also include the stage of the surgical procedure which is being completed by the surgeon.

In some examples, the surgical information may include information regarding the medical status of the patient who is being operated on. This may include information such as the blood pressure of the patient; the oxygen saturation levels of the patient; the abdominal air pressure within the patient, or the like.

<Sources of Additional Information>

Now, as noted above, the additional information may be received by the receiving unit 1002 from one or more sources depending on the situation. In examples, the additional information may be received from one or more sensors in the surgical environment. That is, the additional information may be received from one or more sensors located within the tools being used by the surgeon. Alternatively, position or movement data may be received from orientation information measured by one or more sensors of the computer assisted camera system.

Alternatively, this additional information may be received from analysis of images or video streams from within the surgical environment either internal or external to the patient (this may include images of the patient, surgeons or other features of the operating theatre). Machine vision systems may extract information regarding material classification (to recognise tissue type and/or tissue properties) item identification (tool or organ type, for example) motion recognition (tool movements, tool activity and the like).

Alternatively, the additional information may be extracted from one or more device and/or system interfaces (such as lighting systems, suction devices, operating theatre cameras or the like). Alternatively, the receiving unit 1002 of apparatus 1000 may interface with an operating theatre management unit to obtain relevant patient-external data.

Alternatively, the additional information may be extracted by the first receiving unit 1002 of apparatus 1000 from audio streams captured within the operating theatre (such as conversations between the surgeon and assistants during the surgery). The first receiving unit 1002 may utilize speech recognition technology that enables the apparatus to monitor surgical staff conversations and extract relevant information, for example. The speech recognition technology may enable the apparatus 1000 to detect specific instructions given by the surgeon indicative of the next surgical stage; extract basic keywords from conversations; and/or apply natural language processing to full conversations to obtain all relevant contextual data.

Alternatively, this additional information may be received through manual input received from the surgeon, the medical assistants or support staff. This may include an interface which enables the surgeon and/or medical assistants/support staff to indicate relevant information such as the next surgical stage and/or manually tag items such as tools, organs and other features in the camera's visual feed. The surgical stages may then be used to extract information from a centralised database (using a lookup table or the like) detailing typical surgical workflows, stages, associated procedures and tools used at each stage of the surgical procedure.

Once the additional information and the first image have been received by the receiving unit 1002, the additional information is passed to the determining unit 1004 of apparatus 1000. In some example, the receiving unit 1002 may pass this information directly to determining unit 1004. In other examples, the first receiving unit 1002 may store the additional information in a memory or storage accessible by determining unit 1004.

Determining Unit:

Determining unit 1004 of apparatus 1000 is configured to determine, for the image capture device, in accordance with the additional information and previous viewpoint information of surgical scenes, one or more candidate viewpoints from which to obtain an image of the surgical scene.

These candidate viewpoints are suggested viewpoints within the surgical environment which the image capture device could use in order to provide a clear image of the scene. According to embodiments of the disclosure, these candidate viewpoints are determined on the basis of viewpoints which have been used in previous surgical procedures. As such, the viewpoint information may include position information and/or orientation information of the image capture device (that is, position and/or orientation information of the image capture device as used in previous surgical procedures).

That is, as described above, the additional information received by the first receiving unit 1002 is information which enables the apparatus 1000 to determine information regarding the surgical procedure being performed by the surgeon 804.

Accordingly, in examples, the determining unit 1004 may use this information to query a lookup table providing information about candidate viewpoints for the surgical procedure. The table providing information about candidate viewpoints for the surgical procedure may be constructed based on the operation history of the computer assisted camera system (that is, viewpoints which were used for the image capture device in previous surgeries relating to that surgical procedure, for example).

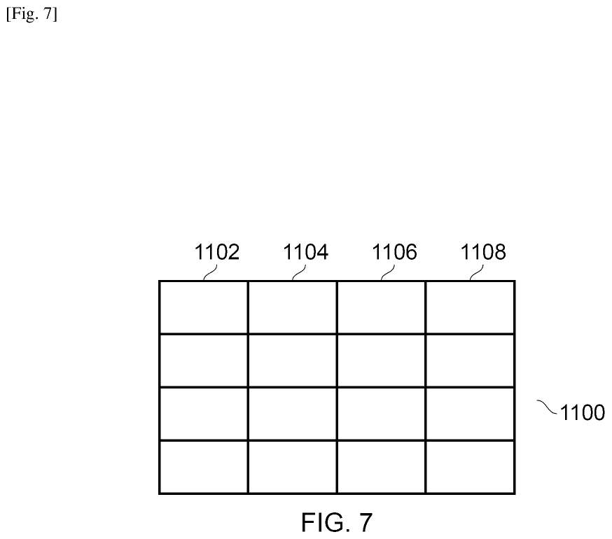

An example lookup table which can be used to determine candidate viewpoints is illustrated with reference to FIG. 7 of the present disclosure.

Lookup query table 1100 may be stored in a storage unit internal to apparatus 1000 or, alternatively, may be stored in an external storage which is accessible by apparatus 1000 (such as an external server). In this specific example, the first column 1102 defines information regarding the surgical procedure (this may also include different entries for different stages of the same surgical procedure (such as the initial, middle and final stage of the surgical procedure)). The determining unit may, on the basis of the surgical procedure determined from the additional information, query the lookup table 1100 in order to determine an entry corresponding to the current surgical procedure (or may perform this lookup on the basis of the additional information itself). Once an entry corresponding to the current surgical procedure has been identified, the determining unit 1004 of apparatus 1000 may then read, from the corresponding rows of subsequent columns 1104, 1106 and 1108, candidate viewpoint information for that surgical procedure.

That is, each of columns 1004, 1106 and 1108 may store information regarding a viewpoint which had been used for the image capture device in previous surgical procedures that match the current procedure.

From this table, the determining unit can therefore determine one or more candidate viewpoints for the current surgical procedure.

That is, in this example lookup query table 1100 enables the determining unit 1004 to extract candidate viewpoints from the autonomous operation history of the computer assisted camera system that are relevant to the current surgical scene. In some examples, candidate viewpoints may be extracted based on previous viewpoints used for comparable surgical procedures (this may include viewpoints used for a different stage of the same surgical procedure, for example).

As described above, lookup query table 1100 may be constructed based on viewpoints used by the computer assisted camera system in previous surgical situations. However, the lookup query table may further be constructed based on viewpoints used by the computer assisted camera system in one or more photorealistic simulations of surgical procedures. Alternatively or in addition, the table may also be constructed based on viewpoints used by other surgeons (either human, or robotic) who have performed the surgical procedure.

In this manner, the lookup table enables the determination unit 1004 to determine candidate viewpoints for the image capture device which may not have been contemplated by the surgeon 804. The candidate viewpoints may therefore be surprising, or unexpected, to the surgeon 804, thus providing the surgeon with a viewpoint they would not previously have contemplated.

Now, it will be appreciated that the example of FIG. 7 is just one example of the determination of the candidate viewpoints which may be performed by determination unit 1004. Any such processing which enables the determination unit 1004 to determine one or more candidate viewpoints based on previous viewpoint preferences relative to the additional information acquired by the first acquiring unit 1002 may be used as required by apparatus 1000.

In this manner, the determination unit 1004 collates viewpoints from previous surgeries as one or more candidate viewpoints for the surgical scene.

In some examples, the determining unit 1004 is configured to analyse the candidate viewpoints in accordance with a predetermined metric, and display the top N candidate viewpoints (top three candidates, for example) to the surgeon for selection. That is, the determining unit may use one or more assessment algorithms in order to assess the viewpoint candidates relative to the current viewpoint, and selector from the candidate viewpoints a subgroup of candidate viewpoints which provide a relative viewpoint advantage to the surgeon. This enables the determining unit 1004 to select a number of candidate viewpoints which provide, or may provide, a viewpoint advantage to the surgeon 804 over the viewpoint from which they are currently operating.

The relative viewpoint advantage to the surgeon may include viewpoints which, from previous surgeries, are known to provide an expanded viewpoint of a specific region of tissue; an expanded viewpoint of a tool being used by the surgeon; an improved recognition of critical features (such as features of the target region, including subsurface veins or a tumour to be removed from the target region); and/or improved lighting conditions (such as less shadow, or less reflection from the tissue surface) or the like.

The selection of N candidate viewpoints may also be performed based on a comparison with the viewpoints to viewpoint preferences of the surgeon 804. This enables the determining unit to determine advantageous candidate viewpoints which would be unlikely to be considered by the surgeon 804, for example.

This assessment is based on the viewpoint information itself (such as the information regarding the candidate viewpoint which has been extracted from the lookup table).

Moreover, in some examples, the advantage assessment unit may be configured to evaluate the candidate viewpoints in accordance with a predetermined metric, and control a display to display, based on the evaluation, at least a subset of the candidate viewpoints. As noted above, the predetermined metric may be based, for example, on a comparison of the candidate viewpoints with one or more viewpoint preferences of the surgeon. In this manner, only a subset of the alternative candidate viewpoints which have been generated are displayed to the surgeon for selection.

Now, returning to the example of FIG. 4 of the present disclosure, the one or more candidate viewpoints could include information regarding candidate locations from which the image capture device could capture an image of the target region 808 of the patient 802. However, the candidate viewpoints may also include information regarding a candidate image capture property of the image capture device. This may include, for example, a candidate imaging type to be used by the image capture device. One of the candidate viewpoints may, for example, be a viewpoint whereby hyperspectral imaging, using spectroscopy, is used to measure varying interactions between the light and radiation within the body.

Another candidate viewpoint may use optical imaging, with visible light illumination, within the body cavity of the patient. Image capture properties, such as the level of zoom or image aperture used by the image capture device, may also be included within the candidate viewpoints determined by the determining unit 1004.

As such, in certain examples, the imaging property of the image capture device may include at least one of an optical system condition of the medical image capture device and/or an image processing condition of the captured image. For example, the optical system condition may include factors such as an optical image zoom, an image focus, an image aperture, an image contrast, an image brightness and/or an imaging type of the image capture device. In contrast, the image processing condition of the captured image may include factors such as a digital image zoom applied to the image and/or factors which relate to the processing of the image (such as image brightness, contrast, saturation, hue or the like).

Moreover, in some examples, a candidate viewpoint may include both static and dynamic viewpoints (that is, a viewpoint from a single location or a viewpoint moving between, or showing, two or more locations of the surgical scene).

Once the list of one or more candidate viewpoints has been determined by the determination unit 1004 the candidate viewpoints are passed to the providing unit 1006 of apparatus 1000 for processing.

Providing Unit:

The providing unit 1006 of apparatus 1000 is configured to provide, in accordance with the first image of the surgical scene, for each of the one or more candidate viewpoints, a simulated image of the surgical scene from that candidate viewpoint.

That is, in examples, the providing unit receives the one or more candidate viewpoints from the determining unit 1004, and the first image from the first receiving unit 1002, and uses this information in order to generate a simulated image of the surgical scene for each candidate viewpoint. These simulated images provide a predicted appearance of how the scene would appear from the candidate viewpoint (and are obtained without actually changing the image capture properties of the image capture device at this stage). These generated images are then provided for selection.

Moreover, in other examples, it will be appreciated that the providing unit of apparatus 1000 may be configured to receive simulated images of the scene which have been previously generated (by an external computing device) and provide those simulated images directly to a surgeon for selection.

Consider again the example situation of FIG. 4 of the present disclosure. In this example the apparatus 1000 has received image 900 (illustrated with reference to FIG. 5 of the present disclosure) as the first image of the scene. This first image of the scene is plagued by a number of reflections off the surface of the tissue which prevent the surgeon 804 from obtaining a clear image of the target region 808. Moreover, in this example, from the additional information of the scene received by the first receiving unit 1002, the determining unit 1004 has determined a selection of three candidate viewpoints which can be used, for a surgical procedure corresponding to the surgical procedure being performed by surgeon 804, which are advantageous in that they are known, from previous surgeries, to reduce the amount of glare or reflections off the surface of the tissue.

Accordingly, in this example, the providing unit 1006 generates a simulated image of the surgical scene as it is predicted that the scene would appear from each of the candidate viewpoints which have been determined. These images are generated in accordance with the first image of the scene 900 which has been received by the first receiving unit. It will be appreciated that the providing unit 1006 generates the simulated images with an aim of reproducing as closely as possible the advantageous robot viewpoints within the context of the current surgical scene.

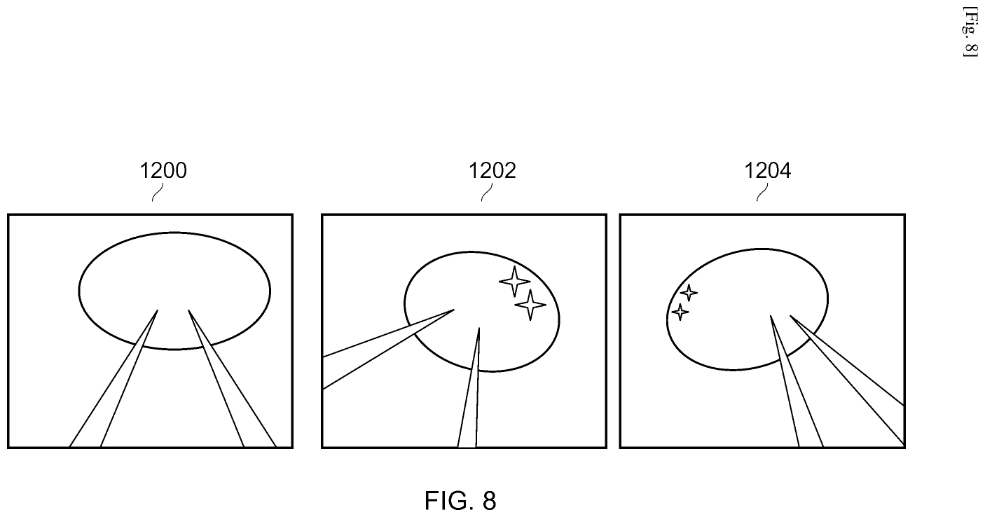

An example illustration of the simulated images for the candidate viewpoints is shown in FIG. 8.

In this example, simulated image 1200 is a simulated image from the first of the candidate viewpoints which has been determined by the determining unit 1004. This first candidate viewpoint is a viewpoint which uses hyperspectral imaging to reduce the reflections from the surface of the tissue. Accordingly, simulated image 1200 shows a prediction of how the target region 808 of the patient would appear when using this hyperspectral imaging.

Simulated image 1202 is a simulated image from the second candidate viewpoint which has been determined by the determining unit 1004. This second candidate viewpoint is a viewpoint where the image capture device captures images from a second physical location within the surgical environment (being a physical location different from the current physical location of the image capture device).

Accordingly, simulated image 1202 shows a prediction of how the target region 808 of the patient would appear when capturing images from this second physical location within the surgical environment.

Finally, simulated image 1204 is a simulated image from the third candidate viewpoint which has been determined by the determining unit 1004. This third candidate viewpoint is a viewpoint where the image capture device captures images from a third physical location (being different to both the current physical location and the physical location of the second candidate viewpoint). Accordingly, simulated image 1204 shows a prediction of how the target region 808 of the patient would appear when capturing images from this third physical location within the surgical environment.

For all three of the simulated images 1200, 1202 and 1204, the amount of glare and reflection from the tissue of the patient is less than that which is present in the current image of the scene 900 (illustrated with reference to FIG. 5 of the present disclosure).

In some examples, the providing unit 1006 may also utilize the additional information received by the first receiving unit 1002 of apparatus 1000 when producing the simulated images of the scene.

Information regarding the surgical environment, such as the respective orientation of elements within the surgical scene, may be used when producing the simulated image of the scene from the candidate viewpoint, for example.

Now, in embodiments of the disclosure, the simulated images of the scene are generated from the first image of the scene, based on the determined candidate viewpoints, using the capability of artificial intelligence systems to simulate an unseen viewpoint of the scene. That is, it is known that an artificial intelligence system can view a scene from a certain first perspective (corresponding to the viewpoint of the first image 900 in this example) and predict what the same scene will look like from another unobserved perspective (corresponding to simulated images 1200, 1202 and 1204 in this example).

In certain examples, this may be implemented, for example, using a machine learning system trained on previous viewpoints of the surgical scene; this can include previous viewpoints of the surgical scene used in previous surgical procedures and can also include one or more viewpoints used in simulations of the surgical scene.

In certain situations, deep learning models (as an example of a machine learning system) may be used in order to generate the simulated images of the scene. These deep learning models are constructed using neural networks. These neural networks include an input layer and an output layer. A number of hidden layers are located between the input layer and the output layer. Each layer includes a number of individual nodes. The nodes of the input layer are connected to the nodes of the first hidden layer. The nodes of the first hidden layer (and each subsequent hidden layer) are connected to the nodes of the following hidden layer. The nodes of the final hidden layer are connected to the nodes of the output layer.

In other words, each of the nodes within a layer connect back to all the nodes in the previous layer of the neural network.

Of course, it will be appreciated that both the number of hidden layers used in the model and the number of individual nodes within each layer may be varied in accordance with the size of the training data and the individual requirements of the simulated image of the scene.

Now, each of the nodes takes a number of inputs, and produces an output. The inputs provided to the node (through connections with the previous layers of the neural network) have weighting factors applied to them.

In a neural network, the input layer receives a number of inputs (which can include the first image of the scene). These inputs are then processed in the hidden layers, using weights that are adjusted during the training. The output layer then produces a prediction from the neural network.

Specifically, during training, the training data may be split into inputs and targets.

The input data is all the data except from the target (being the image of the scene which the neural network is trying to predict).

The input data is then analysed by the neural network during training in order to adjust the weights between the respective nodes of the neural network. In examples, the adjustment of the weights during training may be achieved through linear regression models. However, in other examples, non-linear methods may be implemented in order to adjust the weighting between nodes to train the neural network.

Effectively, during training, the weighting factors applied to the nodes of the neural network are adjusted in order to determine the value of the weighting factors which, for the input data provided, produces the best match to the target data. That is, during training, both the inputs and target outputs are provided. The network then processes the inputs and compares the resulting output against the target data. Differences between the output and the target data are then propagated back through the neural network, causing the neural network to adjust the weights of the respective nodes of the neural network.

Of course, the number of training cycles (or epochs) which are used in order to train the model may vary in accordance with the situation. In some examples, the model may be continuously trained on the training data until the model produces an output within a predetermined threshold of the target data.

Once trained, new input data can then be provided to the input layer of the neural network, which will cause the model to generate (on the basis of the weights applied to each of the nodes of the neural network during training) a predicted output for the given input data.

Of course, it will be appreciated that the present embodiment is not particularly limited to the deep learning models (such as the neural network) and any such machine learning algorithm can be used in accordance with embodiments of the disclosure depending on the situation.

In some examples a Generative Query Network (GQN) may be used in order to generate the simulated images of the scene. In this example, the network collects images from viewpoints within the scene. That is, an image of the surgical scene from the initial location (that is, the first image of the scene) is collected by the GQN. However in other examples, additional images of the scene, depicting how the scene appears from other angles, may be obtained from other image capture devices within the surgical environment.

Alternatively, additional images of the scene may be obtained by the first image capture device during an initial calibration prior to the start of the surgical procedure. As the camera is moved into the initial position to capture images of the target region 808 of the patient, the image capture device may capture images of the surgical scene from slightly different angles (that is, as the image capture device is moved into its initial position). These images may be stored in order to assist in later viewpoint generation. The stored images may range from a small number of frames to a full recording of the motion, depending on the data storage capabilities of the surgical facility, for example. In this manner, images of the scene from a number of viewpoints may be obtained. In certain examples, the apparatus 1000 may be further configured to use this information in order to generate a map of the surgical environment while moving into position. This may be achieved using simultaneous localization and mapping (SLAM) algorithms.

Now, the initial image, or images, obtained by the image capture device during the initial calibration then forms a set of observations for the GQN. Each additional observation (that is, each additional image of the scene from a different viewpoint) enables the GQN to accumulate further evidence regarding the content of the scene.

The GQN, having been trained on the surgical scene, is then able to produce a simulated image of the scene from the one or more candidate viewpoints which have been determined by the determining unit 1004 of the apparatus 1000.

However, it will be appreciated that the GQN is merely one example of an artificial intelligence imaging system which can be used in order to generate the simulated images of the scene in accordance with embodiments of the disclosure. Any other type of artificial intelligence system may be used to generate the simulated image of the candidate viewpoints of the scene as required.

Consider again the example situation described with reference to FIG. 4 of the present disclosure. In this example, once the providing unit 1006 has generated the three simulated images of the surgical scene (images 1200, 1202 and 1204 illustrated in FIG. 8) the providing unit passes those simulated images for display to the surgeon 804.

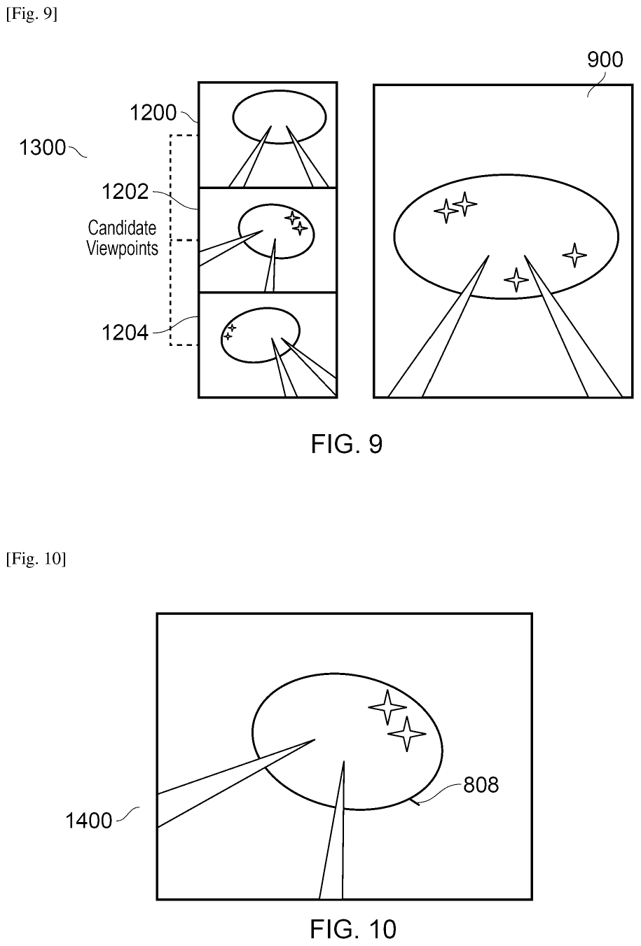

In examples, the providing unit 1006 may provide an interface (the “user interface”) through which the surgeon 804 may interact with the simulations of the candidate viewpoints. An example illustration of the user interface 1300 is shown in FIG. 9 of the present disclosure. User interface 1300 may be displayed on a display screen present in the operating theatre (such as the display screen which is used by the surgeon in order to perform the surgical procedure (that is, the display screen on which the first image of the scene is displayed)). That is, once the simulated images of the scene from the candidate viewpoints have been generated (showing how the scene is predicted to appear from those candidate viewpoints) the apparatus 1000 is configured to provide the simulated images to the surgeon for review.

In this example, the user interface 1300 provided to the surgeon 804 includes a first region which shows the current view of the scene 900 (that is, the first image captured by the image capture device). This is the viewpoint that the surgeon 804 is currently using in order to perform the surgical procedure on the patient. A second region of the user interface is also provided, which displays the simulations of the candidate viewpoints 1200, 1202 and 1204 which have been generated by the providing unit 1006 of apparatus 1000.

As such, from this user interface, the surgeon 804 can see the simulated images of the candidate viewpoints which have been generated by apparatus 1000, and can assess whether these viewpoints provide an advantageous reduction in the glare and reflection which is currently being experienced from the tissue of the target region 808 (as seen in image 900). This enables the surgeon 804 to assess whether a more optimum view of the target region 808 of the patient can be achieved by the image capture device without any delay to the surgical procedure (because, when generating the simulated images of the candidate viewpoints, the image capture device remains in the initial image capture location).

In some examples, apparatus 1000 may autonomously suggest the candidate viewpoints to the surgeon using a user interface 1300 when it determines that an advantage may be gained for the surgeon from a candidate viewpoint.

Alternatively in other examples, the user interface may incorporate a call/request function, whereby the surgeon may instruct the system to generate and provide one or more candidate viewpoints for display.

This may be particularly useful when the surgeon has noticed a deterioration in the image provided by the image capture device, for example.

For each candidate viewpoint that is presented to the surgeon, the providing unit 1006 of apparatus 1000 may also provide one or more pieces of further information regarding the candidate viewpoint. This further information may include information regarding the relationship between the current viewpoint and the candidate viewpoint (this may be communicated through schematic indicating the path the image capture device would take from the current viewpoint to the candidate viewpoint and/or a numerical description of the path the image capture device would take from the current viewpoint to the candidate viewpoint); the purpose of generating the candidate viewpoint (primarily, the advantage gained by adopting the candidate viewpoint (this may include numerical values of anticipated improvements to image quality, for example)).

Of course, while an example user interface is illustrated with reference to FIG. 9 of the present disclosure, the embodiments of the disclosure are not intended to be particularly limited in this regard.

Alternatively, candidate viewpoints may be presented to the Surgeon via, for example, a Picture in Picture (PinP) function integrated with the Surgical Camera display, or via a separate display screen or method.

In fact, any such method which enables the surgeon to view the simulated images which have been generated by the apparatus 1000 may be used in accordance with embodiments of the disclosure.

In this manner, the providing unit 1006 provides realistic visualisations of viewpoints to simulate the appearance of the scene from the one or more candidate viewpoints which have been determined by the determining unit 1004.

Controlling Unit:

At this stage, the image capture device of the computer assisted camera system remains at its initial location (that is, it still captures images from the initial viewpoint of the scene); the simulated images have been produced based upon a prediction of how the scene would appear from that candidate location without moving the camera. However, once a selection of one of the one or more simulated images of the surgical scene has been received, the controlling unit 1008 of apparatus 1000 is configured to control the image capture device to obtain an image of the surgical scene from the candidate viewpoint corresponding to the selection of one of the one or more simulated images of the surgical scene.

The manner of receiving the selection of the one of the one or more simulated images of the surgical scene which have been provided by the providing unit 1006 is not particularly limited.

In examples, controlling unit 1008 is configured to receive, from the surgeon, the medical assistant or the staff, a selection of one of the one or more simulated images of the surgical scene.

That is, in examples, the surgeon can interact with the user interface in order to select one of the simulated images of the candidate viewpoints. This may be a simulated image of a candidate viewpoint for which the surgeon would like the image capture device to move to (such that an actual image of the scene from the candidate viewpoint can be obtained).

That is, the surgeon 804 may use the user interface to accept or select a simulated image of a candidate viewpoint which has been suggested by the system (the “preferred viewpoint”).

Optionally, the surgeon 804 may select multiple preferred viewpoints, which the system may save and apply at the surgeon's request. That is, the surgeon may indicate that they wish to store a viewpoint for use later in the surgical procedure. Alternatively, the surgeon may indicate that they wish a first candidate viewpoint to be adopted for a first time period, followed by a second candidate viewpoint at a later stage of the procedure.

In some examples, the controlling unit may be configured to receive a touch input on the user interface 1300 as a selection of a simulated image of a candidate viewpoint by the surgeon 804. In other examples, the surgeon is able to provide a voice input as a selection of one or more of the simulated images of the candidate viewpoints (such as, “select simulated image number one”, for example).

In fact, any such configuration which enables the controlling unit to receive a selection of one or more of the simulate images of the surgical scene from the surgeon may be used in accordance with embodiments of the disclosure as required.

In certain examples, the controlling unit is configured to determine the candidate image corresponding to the simulated image selected by the surgeon, and perform one or more operations in order to control the image capture device of the computer assisted camera system such that the image capture device is re-configured to capture images of the target region 808 of the patient using the candidate viewpoint corresponding to the simulated image selected by the surgeon.

In certain examples, the control unit may perform camera actuation processing in order to physically move the image capture device to the location corresponding to the selected candidate viewpoint. The image capture device then captures subsequent images of the scene from this actual real world location (corresponding to the candidate location which has been selected by the surgeon). As part of the camera actuation processing, the image capture device may be moved manually by the surgeon or supporting staff, following navigation guidance provided by the apparatus 1000. In this case, navigation guidance may be communicated to the surgeon or supporting staff via the user interface 1300. Alternatively, the image capture device may be moved autonomously by the surgical robot, following verification of the intended motion (as required) by the surgeon. That is, in some examples, the controlling unit may be configured to control the position and/or orientation of an articulated arm supporting the image capture device to control the image capture device to obtain an image of the surgical scene from the candidate viewpoint corresponding to the selection of one of the one or more simulated images of the surgical scene.

In other examples, the control unit may perform camera modulation processing in order to re-configure one or more image capture properties of the image capture device (such as the zoom level) such that the image capture device then captures subsequent images of the scene using this actual real world re-configuration.

Consider again the example situation described with reference to FIG. 4 of the present disclosure. Here, having seen the three simulated images 1200, 1202 and 1204 generated by apparatus 1000, the surgeon 804 has selected candidate viewpoint 1202 as the viewpoint from which they would like the image capture device to capture the subsequent images of the scene. Accordingly, the controlling unit 1008 controls the image capture device of the computer assisted camera system such that subsequent images of the target region 808 are captured from this selected candidate viewpoint.

An example illustration of the real image 1400 captured by the image capture device following a selection of a candidate viewpoint (that is, the selection of simulated image 1202 corresponding to the candidate viewpoint two) is shown in FIG. 10.

That is, in contrast to simulated image 1202 (which is generated by the providing unit 1006 without actuation of the image capture device) and which forms a prediction of how the target image would appear from the third candidate viewpoint, image 1400 shows an image which is actually captured by the image capture device after it has been moved to the third candidate viewpoint. Accordingly, this actual image 1400 can be used by the surgeon 804 to perform the surgical operation on the patient because it relates to an actual image of the target region of the patient.

In the image 1400, the target region 808 of patient 802 is shown. However, in contrast to the first image of the scene 900 (that is, the image of the target region 808 captured from the initial location of the image capture device), image 1400 provides the surgeon with a clear image of the target region 808 of the patient. That is, the amount of glare and reflection received from the tissue of the target region is substantially reduced in image 1400 compared to image 900.

In this manner, the controlling unit of the apparatus 1000 controls the image capture device such that a real image of the scene, corresponding to the selected simulated image, is captured by the image capture device.

### Advantageous Effects

According to embodiments of the disclosure, the apparatus for controlling an image capture device during surgery enables the surgeon to consider multiple alternative viewpoints for a computer assisted camera system during surgery without having to reposition the camera in order to consider alternative viewpoints, thus enabling optimisation of computer assisted camera system viewpoint strategy without causing unnecessary delay to the surgical procedure.

Furthermore, candidate viewpoints may be presented to the surgeon which the surgeon would have been unlikely to contemplate by themselves. These candidate viewpoints may therefore provide surprising benefits which the surgeon had not previously considered, such as an improvement in the surgical performance or a reduction in the duration of the surgery. In particular, embodiments of the disclosure may enable a human surgeon to benefit from viewpoint strategies developed by other human or robotic surgeons.

Of course, the present disclosure is not particularly limited to these advantageous technical effects, there may be others as will become apparent to the skilled person when reading the present disclosure.

Additional Modifications:

While configurations of the apparatus 1000 have been described above with reference to FIGS. 4 to 10 of the present disclosure, it will be appreciated that the embodiments of the disclosure are not limited to this specific example. For example, embodiments of the disclosure may be applied to an image capture device such as an endoscopic image capture device, a telescopic image capture device, a microscopic image capture device or the like as required in accordance with the surgical procedure which is being performed.

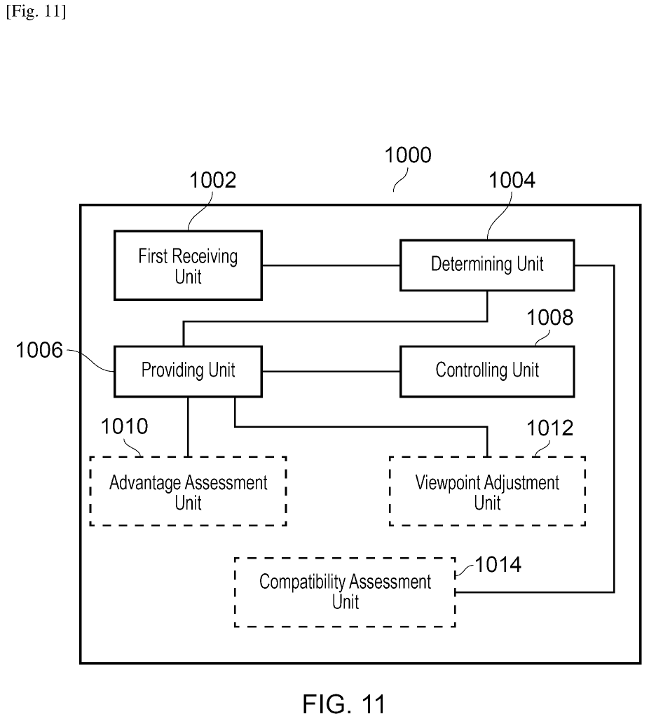

Furthermore, a number of additional modifications to the configuration of the apparatus are described below. FIG. 11 illustrates an apparatus 1000 for controlling an image capture device during surgery according to these embodiments of the disclosure.

<Advantage Assessment Unit>

In some optional examples, the apparatus 1000 may further be configured to include an advantage assessment unit 1010. The advantage assessment unit 1010 may be configured to evaluate one or more quantifiable features of the simulated images of the candidate viewpoints, and arrange the candidate viewpoints in accordance with a result of the evaluation. Candidate viewpoints which the advantage assessment unit evaluates as more advantageous viewpoints for the surgeon, may be arranged in a more prominent position on the display, for example.

That is, the providing unit 1006 may be configured to additionally provide the advantage assessment unit 1010 with the simulated images of the candidate viewpoints, such that the advantage assessment unit can arrange the candidate viewpoints corresponding to those simulated images on the display in accordance with a quantifiable benefit which will be produced for the surgeon. Once the advantage assessment unit 1010 has evaluated the candidate viewpoints, the advantage assessment unit may return this information to the providing unit 1006 such that the providing unit may provide the information regarding the advantageous effect of each candidate viewpoint to the surgeon. Alternatively, or in addition, the information from the advantage assessment unit 1010 may be used by the providing unit 1006 when determining which candidate viewpoints to provide to the surgeon. Alternatively, or in addition, the information from the advantage assessment unit 1010 may be used by the providing unit 1006 when determining the order in which the simulated images corresponding to the candidate viewpoints should be provided to the surgeon.

In examples, the advantage assessment unit 1010 may determine the advantageous effect of each viewpoint relative to the first image received by the first receiving unit 1002 (that is, relative to the current image of the scene obtained by the image capture device).

In examples, the advantage assessment unit 1010 may evaluate the candidate viewpoints based on scores assigned to quantifiable features of the simulated images of the surgical scene. These features may include features such as: a percentage increase in visibility of the surgeon's area of work or key tissue regions; a percentage reduction in light reflection or glare; a percentage increase in the contrast and/or sharpness of the image; a percentage increase in the movement range/degree of movement available to one or more surgical tools within the surgical scene; a reduction in the likelihood of collision between the image capture device and one or more tools within the surgical scene, or the like. A weighting may be applied to each of these features in accordance with the situation, and the simulated image with the highest cumulative score will be evaluated, by the advantage assessment unit 1010, as the most advantage candidate viewpoint for the surgeon. These features may be assessed by the advantage assessment unit 1010 using any suitable image processing techniques as required.

Alternatively, in examples, the unexpectedness of the candidate viewpoint may be factored in the evaluation performed by the advantage assessment unit 1010. That is, the one or more candidate viewpoints determined by the determining unit 1004 (for which simulated images have been generated by the providing unit 1006) may be compared against the viewpoint preferences of the surgeon and/or a viewpoint history unique to that surgeon (indicative of the image capture viewpoints the surgeon typically prefers to use for a given stage of a given surgical procedure). An advantageous viewpoint which has a high degree of contrast to the viewpoints typically selected by the surgeon may be ranked highest by the advantage assessment unit 1010, since these viewpoints are likely to provide the most surprising benefit to the surgeon (being an advantageous viewpoint that the surgeon has not previously contemplated for the surgical procedure).

Furthermore, the candidate viewpoints may further be compared to a database of viewpoints typically used by a global collection of human surgeons for a given stage of a surgical procedure, such that the advantage assessment unit 1010 can determine viewpoints which, while being known to computer assisted surgical systems (such as robotic surgeons) are surprising or unexpected to a large number of human surgeons (and not merely surprising or unexpected to the surgeon who is currently performing the surgical procedure).

In examples, the advantages identified by the advantage assessment unit 1010 which are actually communicated to the surgeon by the providing unit 1006 may vary with the level of experience and/or training of the surgeon. A novice surgeon requiring assistance to find a good viewpoint of the surgical scene may be particularly concerned about collisions between the image capture device and the surgical tools, and may therefore require more working space. A higher weighting factor for working space may therefore be applied by the advantage assessment unit when scoring the candidate viewpoints in this situation.

Alternatively, a surgeon may be using a computer assisted surgical device having more degrees of freedom in the image capture device than computer assisted surgical systems the surgeon has experience with, and therefore the surgeon may not be aware of additional advantageous viewpoints that are possible with the increased range of motion; these additional advantageous viewpoints may be preferentially communicated to the surgeon. That is, a higher weighting factor viewpoints that utilize the enhanced degree of freedom of the image capture device may therefore be applied by the advantage assessment unit when scoring the candidate viewpoints in this situation.

<Viewpoint Adjustment Unit>

In some optional examples, the apparatus 1000 may further be configured to include a viewpoint adjustment unit 1012. The viewpoint adjustment unit may be configured to receive information from the providing unit 1006 regarding the simulated images of the candidate viewpoints that have been provided to the user.

The viewpoint adjustment unit is provided in order to enable the surgeon to modify one or more properties of a selected candidate viewpoint prior to instructing the image capture device to move to that new viewpoint.

In some examples, the viewpoint adjustment unit 1012 may be configured to receive an interaction with a simulated image of the surgical scene and, on the basis of that interaction, update one or more properties of the corresponding candidate viewpoint.

Consider again the example situation described with reference to FIG. 4 of the present disclosure. In this example, the user interface 1300 (illustrated in FIG. 9 of the present disclosure) is provided to the surgeon on a display screen such that the surgeon can perform a selection of the simulated image of a candidate viewpoint as a viewpoint from which the actual images of target region 808 should be obtained.

In this example, when the surgeon performs a selection of a candidate viewpoint, the viewpoint adjustment unit 1012 may be configured to generate a further user interface which, in cooperation with the providing unit 1006, is provided to the surgeon. This further user interface may enable the surgeon to update one or more properties of the corresponding candidate viewpoint.

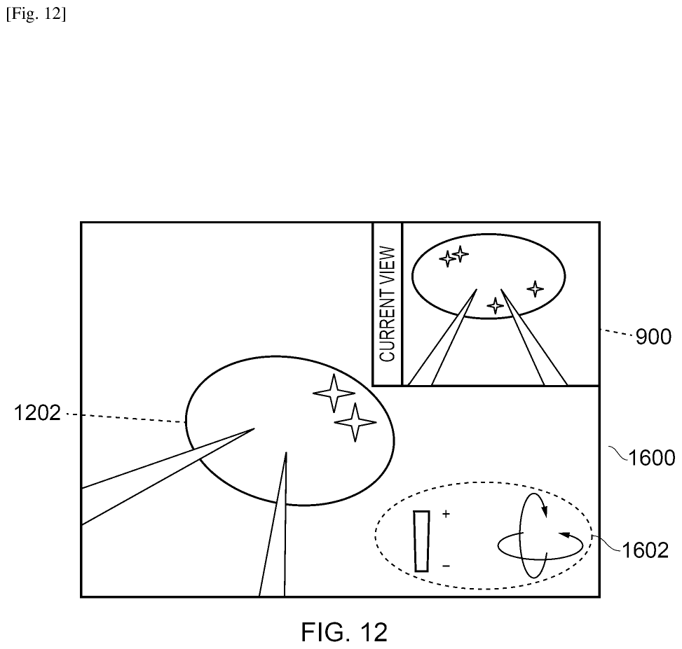

An example of this further user interface 1600 is illustrated in FIG. 12.

Here, the current image of the scene 900 (the first image) is provided to the surgeon in the top portion of the user interface 1600. It is important to continue to provide the current image of the scene to the surgeon such that the surgeon for the safety of the patient and efficiency of the surgical procedure. In addition to this first image 900, user interface 1600 also provides the surgeon with an enhanced view of one of the simulated images which has been produced by the providing unit (being the simulated image which has been selected by the surgeon). In this specific example, the simulated image 1202 has been selected by the surgeon as a candidate viewpoint of interest.

Furthermore, one or more candidate viewpoint adjustment tools 1602 are provided to the surgeon using the user interface 1600. These candidate viewpoint adjustment tools 1602 enable the surgeon to manipulate the simulated image of the candidate viewpoint which has been produced by providing unit 1006. For example, the surgeon may use one of the candidate viewpoint adjustment tools to zoom closer in on the target region. In this situation, the viewpoint adjustment unit is configured to update the simulation of the candidate viewpoint presented to the user and one or more properties of the corresponding candidate viewpoint (being the level of zoom used in the candidate viewpoint in this specific example). Other properties of the candidate viewpoint may include the location of the candidate viewpoint, the aperture of the candidate viewpoint, an image modality of the candidate viewpoint, or the like.

In some embodiments, the providing unit of apparatus 1000 will generate a simulated image of the scene using the updated properties of the candidate viewpoint for provision to the surgeon. That is, in certain examples, the circuitry is configured to receive an interaction with a simulated image of the surgical scene and, on the basis of that interaction, update one or more properties of the corresponding candidate viewpoint and/or the simulated image of the surgical scene.

Accordingly, once the surgeon confirms the selection, the controlling unit 1008 is configured to control the image capture device to capture images from the selected candidate viewpoint as adjusted by the surgeon. Specifically, in this example, the controlling unit controls the image capture device to capture images from the second candidate viewpoint (corresponding to simulated image 1202) with an enhanced level of zoom (corresponding to the adjustment performed by the surgeon).

In other words, the viewpoint adjustment unit 1012 enables the surgeon to manually adjust the selected candidate viewpoint in accordance with their own specific preferences. This enables the surgeon to receive the benefit of the candidate viewpoint, while ensuring that the viewpoint provided by the image capture device is a viewpoint with which the surgeon is comfortable to operate.

<Compatibility Assessment Unit>

In some optional examples, the apparatus 1000 may further be configured to include a compatibility assessment unit 1014. The compatibility assessment unit may receive a list of the candidate viewpoints which have been determined by the determination unit 1004, for example.

In certain examples, the compatibility assessment unit 1014 may be configured to determine the capability of the image capture device to achieve the candidate viewpoints that have been produced by the determining unit and exclude those candidate viewpoints which are unsuitable for the image capture device. That is, owing to restrictions in the working space around the image capture device, the compatibility assessment unit 1014 may determine that the image capture device is not capable of achieving a given candidate viewpoint in a specific surgical situation. A candidate viewpoint which the image capture device is not capable of achieving may then be removed from the list of candidate viewpoints by the compatibility assessment unit 1014 prior to the generation of the simulation of the images of scene obtained from the candidate viewpoints. In this manner, processing resources are not used generating simulated images that cannot be achieved by the image capture device.

In other examples, the compatibility assessment unit 1014 may be configured to perform an assessment of the capability of the candidate viewpoint for use by the surgeon and exclude those candidate viewpoints which are unsuitable for use by the surgeon in the surgical scene. That is, certain candidate viewpoints, while advantageous to a computer assisted surgical system (such as a robotic surgeon) may be too complex for a human surgeon to comprehend. This may be the situation if the viewpoint is a rapidly changing dynamic viewpoint of the scene, for example. In this manner, viewpoints which are impractical for human use may be removed by the compatibility assessment unit 1014 from the list of candidate unit produced by the determining unit of apparatus 1000.

In some examples, the compatibility assessment unit 1014 may be configured to identify certain candidate viewpoints which, whilst in their present form, are incompatible with human surgeons, may be adjusted through one or more modifications such that the candidate viewpoint becomes compatible with human surgeon. For example, certain dynamic robotic viewpoints may be adapted by the compatibility assessment unit 1014 such that the dynamic viewpoint becomes practical for human use. This may be achieved through the compatibility assessment unit 1014 slowing the rate of movement of the image capture device, reducing the number of disparate viewing angles used and/or minimizing frequently switching between different viewing modalities, for example.

In this manner, viewpoints optimized for a computer assisted surgical device may be adapted to increase human surgeon usability of the viewpoint, while still providing a comparable benefit related to the candidate viewpoint to the human surgeon.

### Example Setup

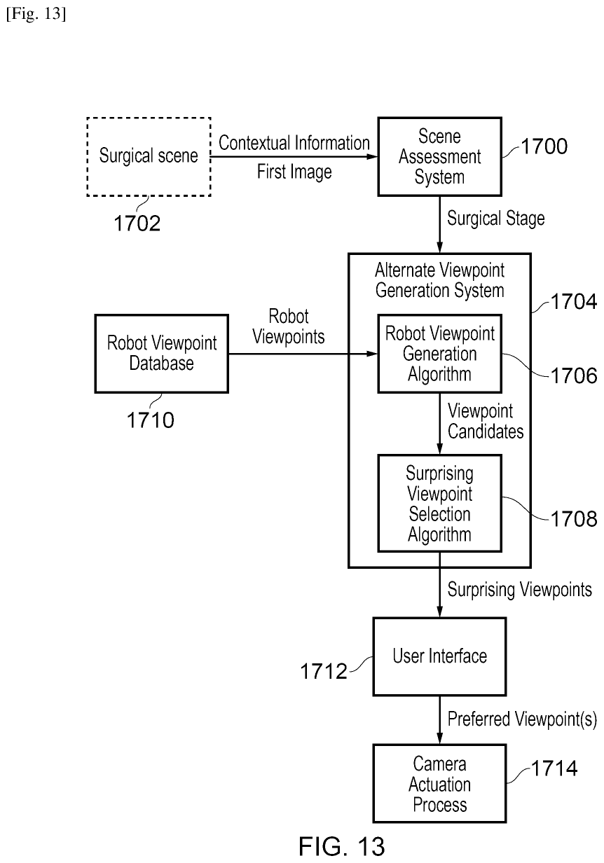

An example setup of a computer assisted surgical system in accordance with embodiments of the present disclosure is illustrated with reference to FIG. 13 of the present disclosure. This example set up may be used in an endoscopic surgical situation (as described with reference to FIG. 1 of the present disclosure) or may, alternatively, be used in a master-slave surgical situation (as described with reference to FIG. 3 of the present disclosure), or may be alternatively used in a surgery using a microscope or an exoscope.

This example setup may be used in order to control an image capture device during surgery in accordance with embodiments of the disclosure.

In this example, a scene assessment system (such as the first receiving unit 1002) receives contextual information and first image information from a surgical scene 1702.

The scene assessment system is configured to use this information which has been received from the surgical scene 1702 in order to determine the surgical stage (that is, the surgical procedure which is being performed by the surgeon, and the stage of that surgical procedure (such as the initial, middle or final stage of the surgical procedure)).

The scene assessment system then provides the information regarding the surgical stage to an alternative viewpoint generating system (such as determining unit 1004 and a providing unit 1006, for example).

The alternative viewpoint generating system 1704 then receives robot viewpoints from a robot viewpoint database. These are viewpoints which robotic surgical systems (which are a form of computer assisted surgical systems) have used in previous surgeries corresponding to the surgery being performed by the surgeon. This is then used, by a robot viewpoint generation algorithm, to generate simulated images of a number of the robot viewpoints (that is, a simulated image of how the surgical scene would appear from certain robot viewpoints retrieved from the robot viewpoint database).

These simulated images are, optionally, passed to a surprising viewpoint selection algorithm which is configured to select a number of the most surprising viewpoints from the viewpoint candidates for provision to the surgeon.

Then, the selected candidate viewpoints are provided to the surgeon using a user interface 1712. As such, the surgeon can see how the image from the image capture device from those selected candidate viewpoints would appear without moving the image capture device and interrupting the surgical procedure.

Upon reception of a selection by the surgeon of one or more preferred viewpoints from the viewpoints which have been displayed on the user interface, a camera actuation unit is configured to control the image capture device of the computer assisted surgical system such that the image capture device is configured to capture subsequent images of the scene from a real world viewpoint corresponding to the virtual candidate viewpoint which has been selected by the surgeon.

In this manner, the surgeon is able to consider multiple alternative viewpoints for a computer assisted camera system during surgery without having to repeatedly reposition the camera in order to consider alternative viewpoints, thus enabling optimisation of computer assisted camera system viewpoint strategy without causing unnecessary delay to the surgical procedure.

Method:

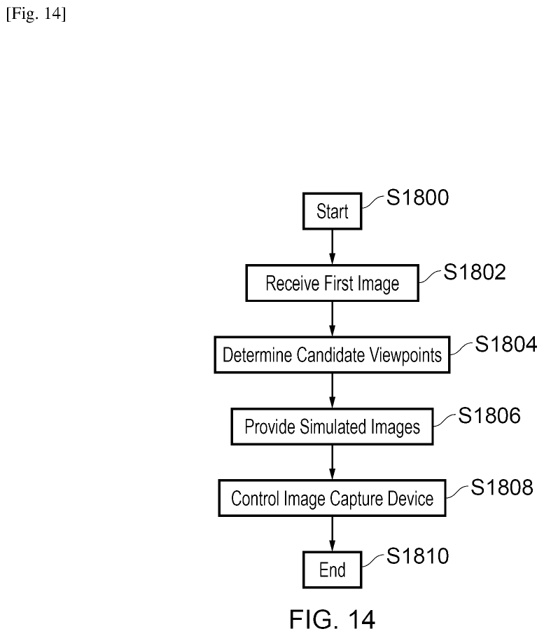

In accordance with embodiments of the disclosure, a method of controlling a medical image capture device during surgery is provided in accordance with embodiments of the disclosure. The method of controlling a medical image capture device is illustrated with reference to FIG. 14 of the present disclosure.

The method starts with step S1800, and proceeds to step S1802.

In step S1802, the method includes receiving a first image of the surgical scene, captured by a medical image capture device from a first viewpoint, and additional information of the scene.

Once the image and additional information have been received, the method proceeds to step S1804.

In step S1804, the method includes determining, for the medical image capture device, in accordance with the additional information and previous viewpoint information of surgical scenes, one or more candidate viewpoints from which to obtain an image of the surgical scene.

Once the candidate viewpoints have been determined, the method proceeds to step S1806.

In step S1806, the method includes providing, in accordance with the first image of the surgical scene, for each of the one or more candidate viewpoints, a simulated image of the surgical scene from that candidate viewpoint.

Once the simulated images of the surgical scene have been provided, the method proceeds to step S1808.

In step S1808, the method includes controlling the medical image capture device to obtain an image of the surgical scene from the candidate viewpoint corresponding to a selection of one of the one or more simulated images of the surgical scene.

The method then proceeds to, and ends with, step S1810.

It will be appreciated that in some situations, once step S1808 has been completed, the method will return to step 1802. In this manner, the desired image capture properties of the image capture device and be continuously or periodically assessed and updated as required.

Computer Device:

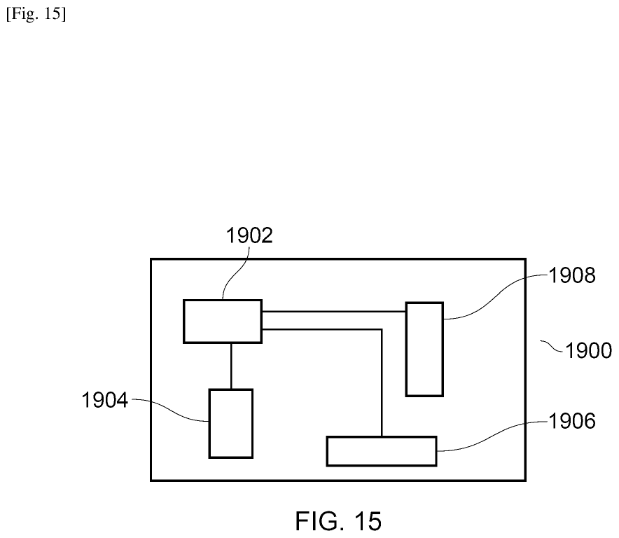

Referring now to FIG. 15, a computing device 1900 according to embodiments of the disclosure is shown. Computing device 1900 may be a computing device for controlling an image capture device during surgery. Typically, the computing device may be a device such as a personal computer or a terminal connected to a server. Indeed, in embodiments, the computing device may also be a server. The computing device 1900 is controlled using a microprocessor or other processing circuitry 1902.

The processing circuitry 1902 may be a microprocessor carrying out computer instructions or may be an Application Specific Integrated Circuit. The computer instructions are stored on storage medium 1904 which may be a magnetically readable medium, optically readable medium or solid state type circuitry.

The storage medium 1904 may be integrated into the computing device 1900 (as illustrated) or may be separate to the computing device 1900 and connected thereto using either a wired or wireless connection.

The computer instructions may be embodied as computer software that contains computer readable code which, when loaded onto the processor circuitry 1902, configures the processor circuitry 1902 of the computing device 1900 to perform a method of controlling an image capture device during surgery according to embodiments of the disclosure. Additionally connected to the processor circuitry 1902, is a user input (not shown). The user input may be a touch screen or may be a mouse or stylist type input device. The user input may also be a keyboard or any combination of these devices.

A network connection 1906 is also coupled to the processor circuitry 1902. The network connection 1906 may be a connection to a Local Area Network or a Wide Area Network such as the Internet or a Virtual Private Network or the like. The network connection 1906 may be connected to a medical device infrastructure allowing the processor circuitry 1902 to communicate with other medical devices in order to obtain relevant data or provide relevant data to the other medical devices. The network connection 1906 may be located behind a firewall or some other form of network security.

Additionally coupled to the processing circuitry 1902, is a display device 1908. The display device 1908, although shown integrated into the computing device 1900, may additionally be separate to the computing device 1900 and may be a monitor or some kind of device allowing the user to visualise the operation of the system. In addition, the display device 1908 may be a printer or some other device allowing relevant information generated by the computing device 1900 to be viewed by the user or by a third party (such as medical support assistants).

Although the foregoing has been described with reference to a “master-slave” robotic system, the disclosure is not so limited. In some instances, the surgical robot may work independently of the human surgeon with the human surgeon being present in a supervisory capacity. Moreover, with endoscopy or laparoscopy, the scopist may be a robot with a human surgeon directing the robot. In embodiments, the robotic system may be a multi-robots surgical system where a main surgeon will use a robotic surgeon and an assistant surgeon will teleoperate assistive robotic arms. The robotic system may be a solo-surgery system which consists of a pair of co-operating and autonomous robotic arms holding the surgical instruments. In this case, the human surgeon may use a master-slave arrangement.

### Example Systems

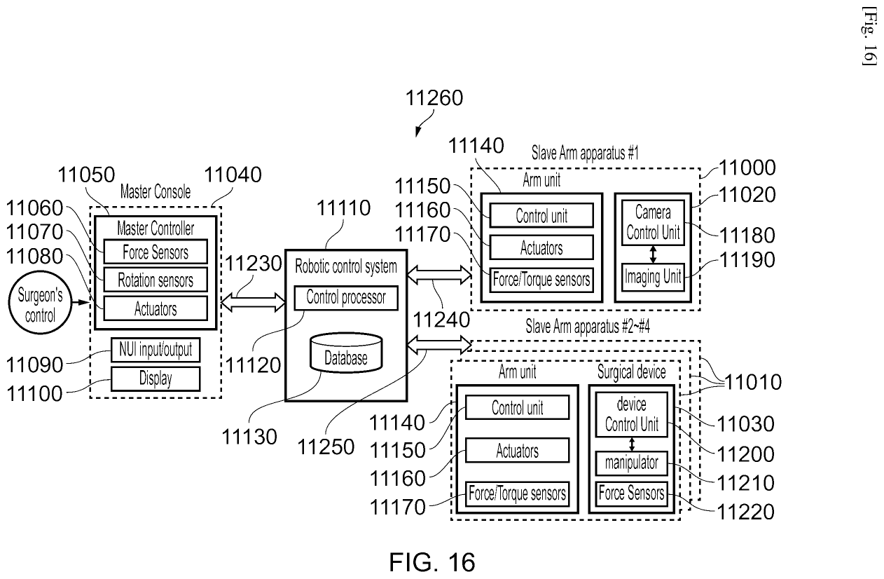

FIG. 16 schematically shows an example of a computer assisted surgery system 11260 to which the present technique is applicable. The computer assisted surgery system is a master slave system incorporating an autonomous arm 11000 and one or more surgeon-controlled arms 11010. The autonomous arm holds an imaging device 11020 (e.g. a medical scope such as an endoscope, microscope or exoscope). The one or more surgeon-controlled arms 11010 each hold a surgical device 11030 (e.g. a cutting tool or the like). The imaging device of the autonomous arm outputs an image of the surgical scene to an electronic display 11100 viewable by the surgeon. The autonomous arm autonomously adjusts the view of the imaging device whilst the surgeon performs the surgery using the one or more surgeon-controlled arms to provide the surgeon with an appropriate view of the surgical scene in real time.

The surgeon controls the one or more surgeon-controlled arms 11010 using a master console 11040. The master console includes a master controller 11050. The master controller 11050 includes one or more force sensors 11060 (e.g. torque sensors), one or more rotation sensors 11070 (e.g. encoders) and one or more actuators 11080. The master console includes an arm (not shown) including one or more joints and an operation portion. The operation portion can be grasped by the surgeon and moved to cause movement of the arm about the one or more joints. The one or more force sensors 11060 detect a force provided by the surgeon on the operation portion of the arm about the one or more joints. The one or more rotation sensors detect a rotation angle of the one or more joints of the arm. The actuator 11080 drives the arm about the one or more joints to allow the arm to provide haptic feedback to the surgeon. The master console includes a natural user interface (NUI) input/output for receiving input information from and providing output information to the surgeon. The NUI input/output includes the arm (which the surgeon moves to provide input information and which provides haptic feedback to the surgeon as output information). The NUI input may also include a voice input, a line of sight input and/or a gesture input.

The master console includes the electronic display 11100 for outputting images captured by the imaging device 11020.

The master console 11040 communicates with each of the autonomous arm 11000 and one or more surgeon-controlled arms 11010 via a robotic control system 11110. The robotic control system is connected to the master console 11040, autonomous arm 11000 and one or more surgeon-controlled arms 11010 by wired or wireless connections 11230, 11240 and 11250. The connections 11230, 11240 and 11250 allow the exchange of wired or wireless signals between the master console, autonomous arm and one or more surgeon-controlled arms.

The robotic control system includes a control processor 11120 and a database 11130. The control processor 11120 processes signals received from the one or more force sensors 11060 and one or more rotation sensors 11070 and outputs control signals in response to which one or more actuators 11160 drive the one or more surgeon controlled arms 11010. In this way, movement of the operation portion of the master console 11040 causes corresponding movement of the one or more surgeon controlled arms.

The control processor 11120 also outputs control signals in response to which one or more actuators 11160 drive the autonomous arm 11000. The control signals output to the autonomous arm are determined by the control processor 11120 in response to signals received from one or more of the master console 11040, one or more surgeon-controlled arms 11010, autonomous arm 11000 and any other signal sources (not shown). The received signals are signals which indicate an appropriate position of the autonomous arm for images with an appropriate view to be captured by the imaging device 11020. The database 11130 stores values of the received signals and corresponding positions of the autonomous arm.

For example, for a given combination of values of signals received from the one or more force sensors 11060 and rotation sensors 11070 of the master controller (which, in turn, indicate the corresponding movement of the one or more surgeon-controlled arms 11010), a corresponding position of the autonomous arm 11000 is set so that images captured by the imaging device 11020 are not occluded by the one or more surgeon-controlled arms 11010.

As another example, if signals output by one or more force sensors 11170 (e.g. torque sensors) of the autonomous arm indicate the autonomous arm is experiencing resistance (e.g. due to an obstacle in the autonomous arm's path), a corresponding position of the autonomous arm is set so that images are captured by the imaging device 11020 from an alternative view (e.g. one which allows the autonomous arm to move along an alternative path not involving the obstacle).

It will be appreciated there may be other types of received signals which indicate an appropriate position of the autonomous arm.

The control processor 11120 looks up the values of the received signals in the database 11130 and retrieves information indicating the corresponding position of the autonomous arm 11000. This information is then processed to generate further signals in response to which the actuators 11160 of the autonomous arm cause the autonomous arm to move to the indicated position.

Each of the autonomous arm 11000 and one or more surgeon-controlled arms 11010 includes an arm unit 11140. The arm unit includes an arm (not shown), a control unit 11150, one or more actuators 11160 and one or more force sensors 11170 (e.g. torque sensors). The arm includes one or more links and joints to allow movement of the arm. The control unit 11150 sends signals to and receives signals from the robotic control system 11110.

In response to signals received from the robotic control system, the control unit 11150 controls the one or more actuators 11160 to drive the arm about the one or more joints to move it to an appropriate position.

For the one or more surgeon-controlled arms 11010, the received signals are generated by the robotic control system based on signals received from the master console 11040 (e.g. by the surgeon controlling the arm of the master console). For the autonomous arm 11000, the received signals are generated by the robotic control system looking up suitable autonomous arm position information in the database 11130.

In response to signals output by the one or more force sensors 11170 about the one or more joints, the control unit 11150 outputs signals to the robotic control system. For example, this allows the robotic control system to send signals indicative of resistance experienced by the one or more surgeon-controlled arms 11010 to the master console 11040 to provide corresponding haptic feedback to the surgeon (e.g. so that a resistance experienced by the one or more surgeon-controlled arms results in the actuators 11080 of the master console causing a corresponding resistance in the arm of the master console). As another example, this allows the robotic control system to look up suitable autonomous arm position information in the database 11130 (e.g. to find an alternative position of the autonomous arm if the one or more force sensors 11170 indicate an obstacle is in the path of the autonomous arm).

The imaging device 11020 of the autonomous arm 11000 includes a camera control unit 11180 and an imaging unit 11190. The camera control unit controls the imaging unit to capture images and controls various parameters of the captured image such as zoom level, exposure value, white balance and the like.

The imaging unit captures images of the surgical scene. The imaging unit includes all components necessary for capturing images including one or more lenses and an image sensor (not shown). The view of the surgical scene from which images are captured depends on the position of the autonomous arm.

The surgical device 11030 of the one or more surgeon-controlled arms includes a device control unit 11200, manipulator 11210 (e.g. including one or more motors and/or actuators) and one or more force sensors 11220 (e.g. torque sensors).

The device control unit 11200 controls the manipulator to perform a physical action (e.g. a cutting action when the surgical device 11030 is a cutting tool) in response to signals received from the robotic control system 11110. The signals are generated by the robotic control system in response to signals received from the master console 11040 which are generated by the surgeon inputting information to the NUI input/output 11090 to control the surgical device. For example, the NUI input/output includes one or more buttons or levers comprised as part of the operation portion of the arm of the master console which are operable by the surgeon to cause the surgical device to perform a predetermined action (e.g. turning an electric blade on or off when the surgical device is a cutting tool).

The device control unit 11200 also receives signals from the one or more force sensors 11220. In response to the received signals, the device control unit provides corresponding signals to the robotic control system 11110 which, in turn, provides corresponding signals to the master console 11040. The master console provides haptic feedback to the surgeon via the NUI input/output 11090. The surgeon therefore receives haptic feedback from the surgical device 11030 as well as from the one or more surgeon-controlled arms 11010. For example, when the surgical device is a cutting tool, the haptic feedback involves the button or lever which operates the cutting tool to give greater resistance to operation when the signals from the one or more force sensors 11220 indicate a greater force on the cutting tool (as occurs when cutting through a harder material, e.g. bone) and to give lesser resistance to operation when the signals from the one or more force sensors 11220 indicate a lesser force on the cutting tool (as occurs when cutting through a softer material, e.g. muscle). The NUI input/output 11090 includes one or more suitable motors, actuators or the like to provide the haptic feedback in response to signals received from the robot control system 11110.

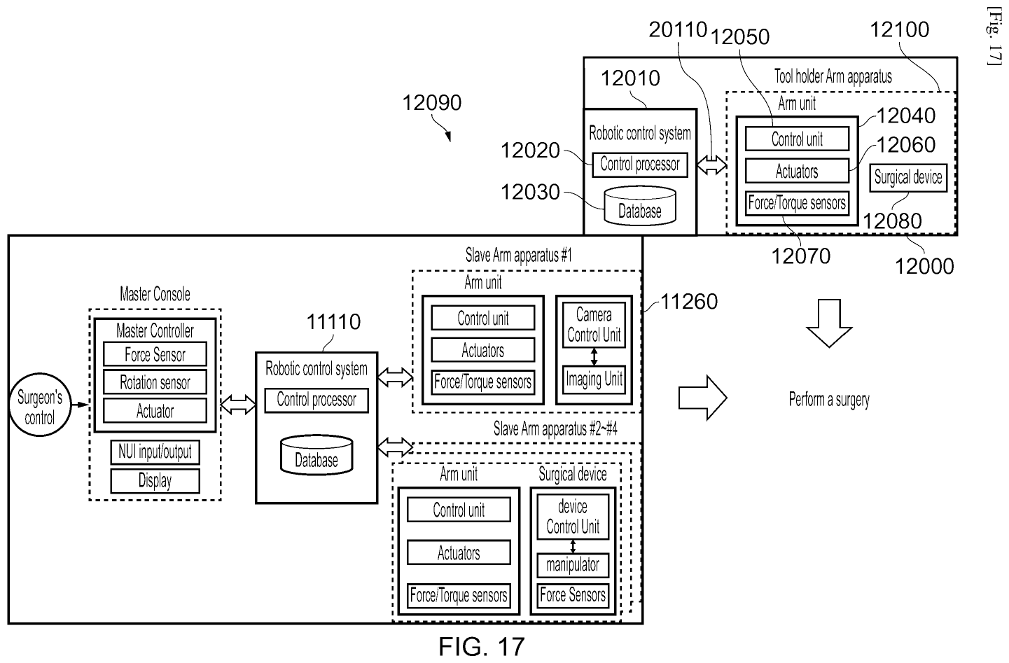

FIG. 17 schematically shows another example of a computer assisted surgery system 12090 to which the present technique is applicable. The computer assisted surgery system 12090 is a surgery system in which the surgeon performs tasks via the master slave system 11260 and a computerised surgical apparatus 12000 performs tasks autonomously.

The master slave system 11260 is the same as FIG. 16 and is therefore not described. The system may, however, be a different system to that of FIG. 16 in alternative embodiments or may be omitted altogether (in which case the system 12090 works autonomously whilst the surgeon performs conventional surgery).

The computerised surgical apparatus 12000 includes a robotic control system 12010 and a tool holder arm apparatus 12100. The tool holder arm apparatus 12100 includes an arm unit 12040 and a surgical device 12080. The arm unit includes an arm (not shown), a control unit 12050, one or more actuators 12060 and one or more force sensors 12070 (e.g. torque sensors). The arm includes one or more joints to allow movement of the arm. The tool holder arm apparatus 12100 sends signals to and receives signals from the robotic control system 12010 via a wired or wireless connection 12110. The robotic control system 12010 includes a control processor 12020 and a database 12030. Although shown as a separate robotic control system, the robotic control system 12010 and the robotic control system 11110 may be one and the same. The surgical device 12080 has the same components as the surgical device 11030. These are not shown in FIG. 17.

In response to control signals received from the robotic control system 12010, the control unit 12050 controls the one or more actuators 12060 to drive the arm about the one or more joints to move it to an appropriate position. The operation of the surgical device 12080 is also controlled by control signals received from the robotic control system 12010. The control signals are generated by the control processor 12020 in response to signals received from one or more of the arm unit 12040, surgical device 12080 and any other signal sources (not shown). The other signal sources may include an imaging device (e.g. imaging device 11020 of the master slave system 11260) which captures images of the surgical scene. The values of the signals received by the control processor 12020 are compared to signal values stored in the database 12030 along with corresponding arm position and/or surgical device operation state information. The control processor 12020 retrieves from the database 12030 arm position and/or surgical device operation state information associated with the values of the received signals. The control processor 12020 then generates the control signals to be transmitted to the control unit 12050 and surgical device 12080 using the retrieved arm position and/or surgical device operation state information.

For example, if signals received from an imaging device which captures images of the surgical scene indicate a predetermined surgical scenario (e.g. via neural network image classification process or the like), the predetermined surgical scenario is looked up in the database 12030 and arm position information and/or surgical device operation state information associated with the predetermined surgical scenario is retrieved from the database. As another example, if signals indicate a value of resistance measured by the one or more force sensors 12070 about the one or more joints of the arm unit 12040, the value of resistance is looked up in the database 12030 and arm position information and/or surgical device operation state information associated with the value of resistance is retrieved from the database (e.g. to allow the position of the arm to be changed to an alternative position if an increased resistance corresponds to an obstacle in the arm's path). In either case, the control processor 12020 then sends signals to the control unit 12050 to control the one or more actuators 12060 to change the position of the arm to that indicated by the retrieved arm position information and/or signals to the surgical device 12080 to control the surgical device 12080 to enter an operation state indicated by the retrieved operation state information (e.g. turning an electric blade to an “on” state or “off” state if the surgical device 12080 is a cutting tool).

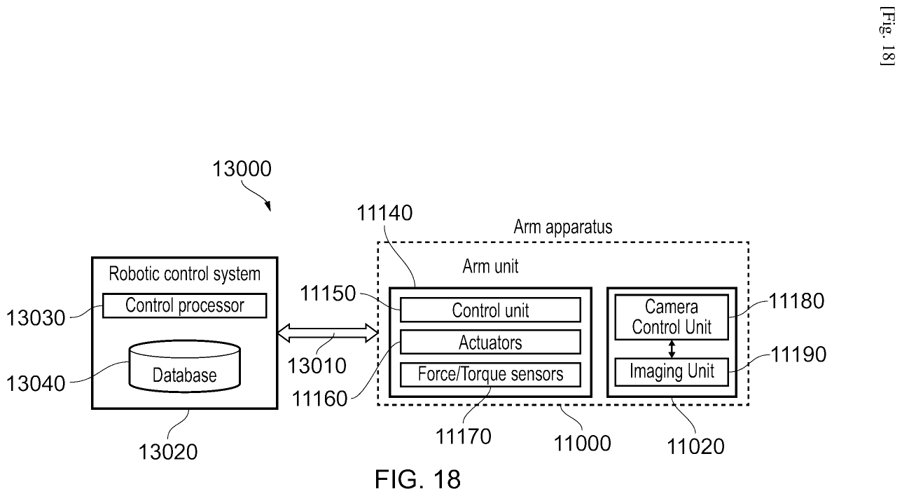

FIG. 18 schematically shows another example of a computer assisted surgery system 13000 to which the present technique is applicable. The computer assisted surgery system 13000 is a computer assisted medical scope system in which an autonomous arm 11000 holds an imaging device 11020 (e.g. a medical scope such as an endoscope, microscope or exoscope). The imaging device of the autonomous arm outputs an image of the surgical scene to an electronic display (not shown) viewable by the surgeon. The autonomous arm autonomously adjusts the view of the imaging device whilst the surgeon performs the surgery to provide the surgeon with an appropriate view of the surgical scene in real time. The autonomous arm 11000 is the same as that of FIG. 16 and is therefore not described. However, in this case, the autonomous arm is provided as part of the standalone computer assisted medical scope system 13000 rather than as part of the master slave system 11260 of FIG. 16. The autonomous arm 11000 can therefore be used in many different surgical setups including, for example, laparoscopic surgery (in which the medical scope is an endoscope) and open surgery.

The computer assisted medical scope system 13000 also includes a robotic control system 13020 for controlling the autonomous arm 11000. The robotic control system 13020 includes a control processor 13030 and a database 13040. Wired or wireless signals are exchanged between the robotic control system 13020 and autonomous arm 11000 via connection 13010.

In response to control signals received from the robotic control system 13020, the control unit 11150 controls the one or more actuators 11160 to drive the autonomous arm 11000 to move it to an appropriate position for images with an appropriate view to be captured by the imaging device 11020. The control signals are generated by the control processor 13030 in response to signals received from one or more of the arm unit 11140, imaging device 11020 and any other signal sources (not shown). The values of the signals received by the control processor 13030 are compared to signal values stored in the database 13040 along with corresponding arm position information. The control processor 13030 retrieves from the database 13040 arm position information associated with the values of the received signals. The control processor 13030 then generates the control signals to be transmitted to the control unit 11150 using the retrieved arm position information.

For example, if signals received from the imaging device 11020 indicate a predetermined surgical scenario (e.g. via neural network image classification process or the like), the predetermined surgical scenario is looked up in the database 13040 and arm position information associated with the predetermined surgical scenario is retrieved from the database. As another example, if signals indicate a value of resistance measured by the one or more force sensors 11170 of the arm unit 11140, the value of resistance is looked up in the database 12030 and arm position information associated with the value of resistance is retrieved from the database (e.g. to allow the position of the arm to be changed to an alternative position if an increased resistance corresponds to an obstacle in the arm's path). In either case, the control processor 13030 then sends signals to the control unit 11150 to control the one or more actuators 1116 to change the position of the arm to that indicated by the retrieved arm position information.

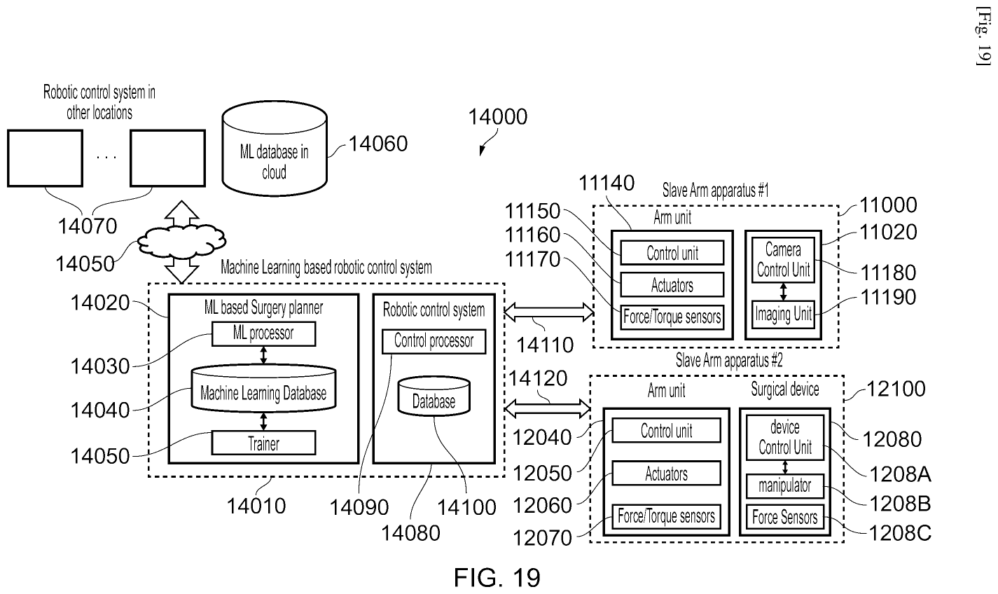

FIG. 19 schematically shows another example of a computer assisted surgery system 14000 to which the present technique is applicable. The system includes one or more autonomous arms 11000 with an imaging unit 11020 and one or more autonomous arms 12100 with a surgical device 12100. The one or more autonomous arms 11000 and one or more autonomous arms 12100 are the same as those previously described.

Each of the autonomous arms 11000 and 12100 is controlled by a robotic control system 14080 including a control processor 14090 and database 14100. Wired or wireless signals are transmitted between the robotic control system 14080 and each of the autonomous arms 11000 and 12100 via connections 14110 and 14120, respectively. The robotic control system 14080 performs the functions of the previously described robotic control systems 11110 and/or 13020 for controlling each of the autonomous arms 11000 and performs the functions of the previously described robotic control system 12010 for controlling each of the autonomous arms 12100.

The autonomous arms 11000 and 12100 perform at least a part of the surgery completely autonomously (e.g. when the system 14000 is an open surgery system). The robotic control system 14080 controls the autonomous arms 11000 and 12100 to perform predetermined actions during the surgery based on input information indicative of the current stage of the surgery and/or events happening in the surgery. For example, the input information includes images captured by the image capture device 11000. The input information may also include sounds captured by a microphone (not shown), detection of in-use surgical instruments based on motion sensors comprised with the surgical instruments (not shown) and/or any other suitable input information.

The input information is analysed using a suitable machine learning (ML) algorithm (e.g. a suitable artificial neural network) implemented by machine learning based surgery planning apparatus 14020. The planning apparatus 14020 includes a machine learning processor 14030, a machine learning database 14040 and a trainer 14050.

The machine learning database 14040 includes information indicating classifications of surgical stages (e.g. making an incision, removing an organ or applying stitches) and/or surgical events (e.g. a bleed or a patient parameter falling outside a predetermined range) and input information known in advance to correspond to those classifications (e.g. one or more images captured by the imaging device 11020 during each classified surgical stage and/or surgical event). The machine learning database 14040 is populated during a training phase by providing information indicating each classification and corresponding input information to the trainer 14050. The trainer 14050 then uses this information to train the machine learning algorithm (e.g. by using the information to determine suitable artificial neural network parameters). The machine learning algorithm is implemented by the machine learning processor 14030.

Once trained, previously unseen input information (e.g. newly captured images of a surgical scene) can be classified by the machine learning algorithm to determine a surgical stage and/or surgical event associated with that input information. The machine learning database also includes action information indicating the actions to be undertaken by each of the autonomous arms 11000 and 12100 in response to each surgical stage and/or surgical event stored in the machine learning database (e.g. controlling the autonomous arm 12100 to make the incision at the relevant location for the surgical stage “making an incision” and controlling the autonomous arm 12100 to perform an appropriate cauterisation for the surgical event “bleed”). The machine learning based surgery planner 14020 is therefore able to determine the relevant action to be taken by the autonomous arms 11000 and/or 12100 in response to the surgical stage and/or surgical event classification output by the machine learning algorithm. Information indicating the relevant action is provided to the robotic control system 14080 which, in turn, provides signals to the autonomous arms 11000 and/or 12100 to cause the relevant action to be performed.

The planning apparatus 14020 may be included within a control unit 14010 with the robotic control system 14080, thereby allowing direct electronic communication between the planning apparatus 14020 and robotic control system 14080. Alternatively or in addition, the robotic control system 14080 may receive signals from other devices 14070 over a communications network 14050 (e.g. the internet). This allows the autonomous arms 11000 and 12100 to be remotely controlled based on processing carried out by these other devices 14070. In an example, the devices 14070 are cloud servers with sufficient processing power to quickly implement complex machine learning algorithms, thereby arriving at more reliable surgical stage and/or surgical event classifications. Different machine learning algorithms may be implemented by different respective devices 14070 using the same training data stored in an external (e.g. cloud based) machine learning database 14060 accessible by each of the devices. Each device 14070 therefore does not need its own machine learning database (like machine learning database 14040 of planning apparatus 14020) and the training data can be updated and made available to all devices 14070 centrally. Each of the devices 14070 still includes a trainer (like trainer 14050) and machine learning processor (like machine learning processor 14030) to implement its respective machine learning algorithm.

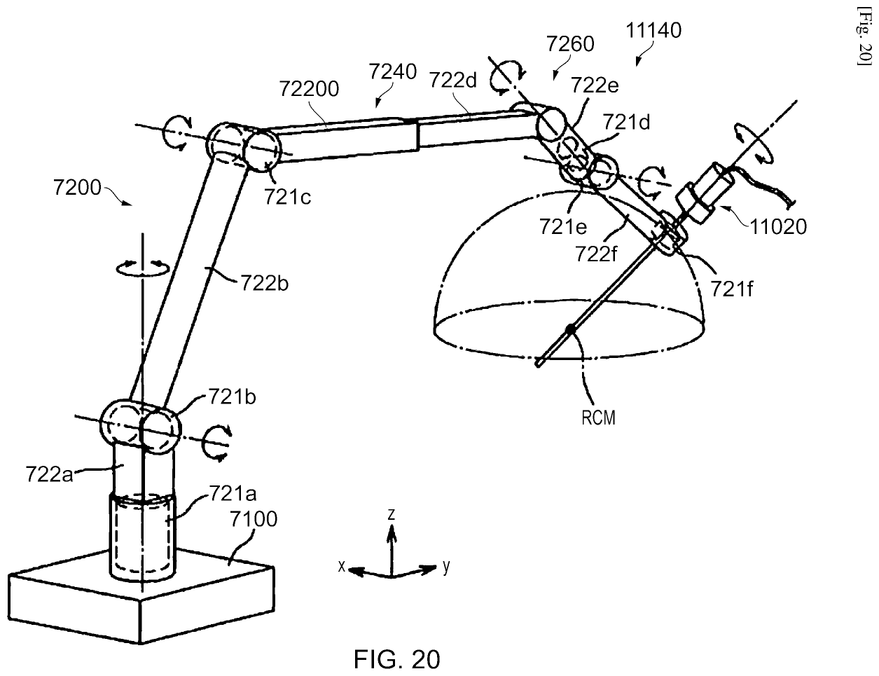

FIG. 20 shows an example of the arm unit 11140. The arm unit 12040 is configured in the same way. In this example, the arm unit 11140 supports an endoscope as an imaging device 11020. However, in another example, a different imaging device 11020 or surgical device 11030 (in the case of arm unit 11140) or 12080 (in the case of arm unit 12040) is supported.

The arm unit 11140 includes a base 7100 and an arm 7200 extending from the base 7100. The arm 7200 includes a plurality of active joints 721a to 721f and supports the endoscope 11020 at a distal end of the arm 7200. The links 722a to 722f are substantially rod-shaped members. Ends of the plurality of links 722a to 722f are connected to each other by active joints 721a to 721f, a passive slide mechanism 7240 and a passive joint 7260. The base unit 7100 acts as a fulcrum so that an arm shape extends from the base 7100.

A position and a posture of the endoscope 11020 are controlled by driving and controlling actuators provided in the active joints 721a to 721f of the arm 7200. According to this example, a distal end of the endoscope 11020 is caused to enter a patient's body cavity, which is a treatment site, and captures an image of the treatment site. However, the endoscope 11020 may instead be another device such as another imaging device or a surgical device. More generally, a device held at the end of the arm 7200 is referred to as a distal unit or distal device.

Here, the arm unit 7200 is described by defining coordinate axes as illustrated in FIG. 14 as follows.

Furthermore, a vertical direction, a longitudinal direction, and a horizontal direction are defined according to the coordinate axes. In other words, a vertical direction with respect to the base 7100 installed on the floor surface is defined as a z-axis direction and the vertical direction. Furthermore, a direction orthogonal to the z axis, the direction in which the arm 7200 is extended from the base 7100 (in other words, a direction in which the endoscope 11020 is positioned with respect to the base 7100) is defined as a y-axis direction and the longitudinal direction. Moreover, a direction orthogonal to the y-axis and z-axis is defined as an x-axis direction and the horizontal direction.

The active joints 721a to 721f connect the links to each other to be rotatable. The active joints 721a to 721f have the actuators, and have each rotation mechanism that is driven to rotate about a predetermined rotation axis by drive of the actuator. As the rotational drive of each of the active joints 721a to 721f is controlled, it is possible to control the drive of the arm 7200, for example, to extend or contract (fold) the arm unit 7200.

The passive slide mechanism 7240 is an aspect of a passive form change mechanism, and connects the link 722c and the link 722d to each other to be movable forward and rearward along a predetermined direction. The passive slide mechanism 7240 is operated to move forward and rearward by, for example, a user, and a distance between the active joint 721c at one end side of the link 722c and the passive joint 7260 is variable. With the configuration, the whole form of the arm unit 7200 can be changed.

The passive joint 7360 is an aspect of the passive form change mechanism, and connects the link 722d and the link 722e to each other to be rotatable. The passive joint 7260 is operated to rotate by, for example, the user, and an angle formed between the link 722d and the link 722e is variable. With the configuration, the whole form of the arm unit 7200 can be changed.

In an embodiment, the arm unit 11140 has the six active joints 721a to 721f, and six degrees of freedom are realized regarding the drive of the arm 7200. That is, the passive slide mechanism 7260 and the passive joint 7260 are not objects to be subjected to the drive control while the drive control of the arm unit 11140 is realized by the drive control of the six active joints 721a to 721f.

Specifically, as illustrated in FIG. 14, the active joints 721a, 721d, and 721f are provided so as to have each long axis direction of the connected links 722a and 722e and a capturing direction of the connected endoscope 11020 as a rotational axis direction. The active joints 721b, 721c, and 721e are provided so as to have the x-axis direction, which is a direction in which a connection angle of each of the connected links 722a to 722c, 722e, and 722f and the endoscope 11020 is changed within a y-z plane (a plane defined by the y axis and the z axis), as a rotation axis direction. In this manner, the active joints 721a, 721d, and 721f have a function of performing so-called yawing, and the active joints 421b, 421c, and 421e have a function of performing so-called pitching.

Since the six degrees of freedom are realized with respect to the drive of the arm 7200 in the arm unit 11140 the endoscope 11020 can be freely moved within a movable range of the arm 7200. FIG. 14 illustrates a hemisphere as an example of the movable range of the endoscope 11020. Assuming that a central point RCM (remote center of motion) of the hemisphere is a capturing centre of a treatment site captured by the endoscope 11020, it is possible to capture the treatment site from various angles by moving the endoscope 11020 on a spherical surface of the hemisphere in a state where the capturing centre of the endoscope 11020 is fixed at the centre point of the hemisphere.

Embodiments of the present disclosure are also defined by the following numbered clauses:

(1)

A system for controlling a medical image capture device during surgery, the system including: circuitry configured to receive a first image of the surgical scene, captured by the medical image capture device from a first viewpoint, and additional information of the scene; determine, for the medical image capture device, in accordance with the additional information and previous viewpoint information of surgical scenes, one or more candidate viewpoints from which to obtain an image of the surgical scene; provide, in accordance with the first image of the surgical scene, for each of the one or more candidate viewpoints, a simulated image of the surgical scene from the candidate viewpoint; control the medical image capture device to obtain an image of the surgical scene from the candidate viewpoint corresponding to a selection of one of the one or more simulated images of the surgical scene.

(2)

The system according to Clause 1, wherein the circuitry is further configured to perform an assessment of the capability of the candidate viewpoint for use by a user and exclude those candidate viewpoints which are unsuitable for use by the user in the surgical scene.

(3)

The system according to any preceding Clause, wherein the circuitry is further configured to: provide the one or more simulated images of the surgical scene for display to a user; receive, from the user, a selection of one of the one or more simulated images of the surgical scene.

(4)

The system according to any preceding Clause, wherein the circuitry is further configured to control the position and/or orientation of an articulated arm supporting the medical image capture device to control the medical image capture device to obtain an image of the surgical scene from the candidate viewpoint corresponding to the selection of one of the one or more simulated images of the surgical scene.

(5)

The system according to any preceding Clause, wherein the circuitry is configured to analyse the candidate viewpoints in accordance with a predetermined metric, and display the top N candidate viewpoints to the user for selection.

(6)

The system according to Clause 5 wherein the circuitry is configured to analyse the candidate viewpoints in accordance with a comparison of the candidate viewpoints with one or more viewpoint preferences of the user as the predetermined metric.

(7)

The system according to any preceding Clause, wherein the circuitry is configured to evaluate the candidate viewpoints in accordance with a predetermined metric, and control a display to display, based on the evaluation, at least a subset of the candidate viewpoints.

(8)

The system according to Clause 5, 6 or 7, wherein the circuitry is configured to evaluate one or more quantifiable features of the simulated images and arrange the candidate viewpoints in accordance with a result of the evaluation as the predetermined metric.

(9)

The system according to any preceding Clause, wherein the circuitry is configured to determine the capability of the image capture device to achieve the candidate viewpoints and exclude those candidate viewpoints which are unsuitable for the image capture device.

(10)

The system according to any preceding Clause, wherein, the additional information received by the circuitry includes surgical and/or environmental data of the surgical scene.

(11)

The system according to Clause 10, wherein the surgical and/or environmental data of the surgical scene includes at least one of: surgical information indicative of the status of the surgery; position data of objects in the surgical environment; movement data of objects in the surgical environment; information regarding a type of surgical tool used by the user; lighting information regarding the surgical environment; and patient information indicative of the status of the patient.

(12)

The system according to any preceding Clause, wherein the circuitry is configured to receive an interaction with a simulated image of the surgical scene and, on the basis of that interaction, update one or more properties of the corresponding candidate viewpoint and/or the simulated image of the surgical scene.

(13)

The system according to any preceding Clause, wherein the circuitry is configured to determine the viewpoint information in accordance with at least one of previous viewpoints selected by the apparatus for a surgical scene corresponding to the additional information and previous viewpoints used by other users for a surgical scene corresponding to the additional information.

(14)

The system according to Clause 12, wherein the viewpoint information includes a position information and/or orientation information of the image capture device.

(15)

The system according to any preceding Clause, wherein the circuitry is configured to use a machine learning system trained on previous viewpoints of the surgical scene to generate the simulated images of the candidate viewpoints.

(16)

The system according to any preceding Clause, wherein the circuitry is configured to control the image capture device to obtain an image from a number of discrete predetermined locations within the surgical scene as an initial calibration in order to obtain the previous viewpoints of the surgical scene.

(17)

The system according to any preceding Clause, wherein the candidate viewpoints include at least one of a candidate location and/or a candidate imaging property of the image capture device.

(18)

The system according to Clause 17, wherein the imaging property includes at least one of an image zoom, an image focus, an image aperture, an image contrast, an image brightness, and/or an imaging type of the image capture device.

(19)

The system according to any preceding Clause, wherein the circuitry is configured to receive at least one of a touch input, a keyboard input or a voice input as the selection of the one of the one or more simulated images of the surgical scene.

(20)

A method of controlling a medical image capture device during surgery, the method comprising:

receiving a first image of the surgical scene, captured by the medical image capture device from a first viewpoint, and additional information of the scene;

determining, for the medical image capture device, in accordance with the additional information and previous viewpoint information of surgical scenes, one or more candidate viewpoints from which to obtain an image of the surgical scene;

providing, in accordance with the first image of the surgical scene, for each of the one or more candidate viewpoints, a simulated image of the surgical scene from the candidate viewpoint;

controlling the medical image capture device to obtain an image of the surgical scene from the candidate viewpoint corresponding to a selection of one of the one or more simulated images of the surgical scene.

(21)

A computer program product including instructions which, when the program is executed by a computer, cause the computer to carry out a method of controlling a medical image capture device during surgery, the method comprising:

receiving a first image of the surgical scene, captured by the medical image capture device from a first viewpoint, and additional information of the scene;

determining, for the medical image capture device, in accordance with the additional information and previous viewpoint information of surgical scenes, one or more candidate viewpoints from which to obtain an image of the surgical scene;

providing, in accordance with the first image of the surgical scene, for each of the one or more candidate viewpoints, a simulated image of the surgical scene from the candidate viewpoint;

controlling the medical image capture device to obtain an image of the surgical scene from the candidate viewpoint corresponding to a selection of one of the one or more simulated images of the surgical scene.

Obviously, numerous modifications and variations of the present disclosure are possible in light of the above teachings. It is therefore to be understood that within the scope of the appended claims, the disclosure may be practiced otherwise than as specifically described herein.

In so far as embodiments of the disclosure have been described as being implemented, at least in part, by software-controlled data processing apparatus, it will be appreciated that a non-transitory machine-readable medium carrying such software, such as an optical disk, a magnetic disk, semiconductor memory or the like, is also considered to represent an embodiment of the present disclosure.

It will be appreciated that the above description for clarity has described embodiments with reference to different functional units, circuitry and/or processors. However, it will be apparent that any suitable distribution of functionality between different functional units, circuitry and/or processors may be used without detracting from the embodiments.

Described embodiments may be implemented in any suitable form including hardware, software, firmware or any combination of these. Described embodiments may optionally be implemented at least partly as computer software running on one or more data processors and/or digital signal processors. The elements and components of any embodiment may be physically, functionally and logically implemented in any suitable way. Indeed the functionality may be implemented in a single unit, in a plurality of units or as part of other functional units. As such, the disclosed embodiments may be implemented in a single unit or may be physically and functionally distributed between different units, circuitry and/or processors.

Although the present disclosure has been described in connection with some embodiments, it is not intended to be limited to the specific form set forth herein. Additionally, although a feature may appear to be described in connection with particular embodiments, one skilled in the art would recognize that various features of the described embodiments may be combined in any manner suitable to implement the technique.

1. A system for controlling a medical image capture device during surgery, the system including: circuitry configured to
receive a first image of the surgical scene, captured by the medical image capture device from a first viewpoint, and additional information of the scene;
determine, for the medical image capture device, in accordance with the additional information and previous viewpoint information of surgical scenes, one or more candidate viewpoints from which to obtain an image of the surgical scene;
provide, in accordance with the first image of the surgical scene, for each of the one or more candidate viewpoints, a simulated image of the surgical scene from the candidate viewpoint;
control the medical image capture device to obtain an image of the surgical scene from the candidate viewpoint corresponding to a selection of one of the one or more simulated images of the surgical scene.

2. The system according to claim 1, wherein the circuitry is further configured to perform an assessment of the capability of the candidate viewpoint for use by a user and exclude those candidate viewpoints which are unsuitable for use by the user in the surgical scene.

3. The system according to claim 1, wherein the circuitry is further configured to:
provide the one or more simulated images of the surgical scene for display to a user;
receive, from the user, a selection of one of the one or more simulated images of the surgical scene.

4. The system according to claim 1, wherein the circuitry is further configured to control the position and/or orientation of an articulated arm supporting the medical image capture device to control the medical image capture device to obtain an image of the surgical scene from the candidate viewpoint corresponding to the selection of one of the one or more simulated images of the surgical scene.

5. The system according to claim 1, wherein the circuitry is configured to analyse the candidate viewpoints in accordance with a predetermined metric, and display the top N candidate viewpoints to the user for selection.

6. The system according to claim 5 wherein the circuitry is configured to analyse the candidate viewpoints in accordance with a comparison of the candidate viewpoints with one or more viewpoint preferences of the user as the predetermined metric.

7. The system according to claim 1, wherein the circuitry is configured to evaluate the candidate viewpoints in accordance with a predetermined metric, and control a display to display, based on the evaluation, at least a subset of the candidate viewpoints.

8. The system according to claim 5, wherein the circuitry is configured to evaluate one or more quantifiable features of the simulated images and arrange the candidate viewpoints in accordance with a result of the evaluation as the predetermined metric.

9. The system according to claim 1, wherein the circuitry is configured to determine the capability of the image capture device to achieve the candidate viewpoints and exclude those candidate viewpoints which are unsuitable for the image capture device.

10. The system according to claim 1, wherein, the additional information received by the circuitry includes surgical and/or environmental data of the surgical scene.

11. The system according to claim 10, wherein the surgical and/or environmental data of the surgical scene includes at least one of: surgical information indicative of the status of the surgery; position data of objects in the surgical environment; movement data of objects in the surgical environment; information regarding a type of surgical tool used by the user; lighting information regarding the surgical environment; and patient information indicative of the status of the patient.

12. The system according to claim 1, wherein the circuitry is configured to receive an interaction with a simulated image of the surgical scene and, on the basis of that interaction, update one or more properties of the corresponding candidate viewpoint and/or the simulated image of the surgical scene.

13. The system according to claim 1, wherein the circuitry is configured to determine the viewpoint information in accordance with at least one of previous viewpoints selected by the apparatus for a surgical scene corresponding to the additional information and previous viewpoints used by other users for a surgical scene corresponding to the additional information.

14. The system according to claim 12, wherein the viewpoint information includes a position information and/or orientation information of the image capture device.

15. The system according to claim 1, wherein the circuitry is configured to use a machine learning system trained on previous viewpoints of the surgical scene to generate the simulated images of the candidate viewpoints.

16. The system according to claim 1, wherein the circuitry is configured to control the image capture device to obtain an image from a number of discrete predetermined locations within the surgical scene as an initial calibration in order to obtain the previous viewpoints of the surgical scene.

17. The system according to claim 1, wherein the candidate viewpoints include at least one of a candidate location and/or a candidate imaging property of the image capture device.

18. The system according to claim 17, wherein the imaging property includes at least one of an image zoom, an image focus, an image aperture, an image contrast, an image brightness, and/or an imaging type of the image capture device.

19. The system according to claim 1, wherein the circuitry is configured to receive at least one of a touch input, a keyboard input or a voice input as the selection of the one of the one or more simulated images of the surgical scene.

20. A method of controlling a medical image capture device during surgery, the method comprising:
receiving a first image of the surgical scene, captured by the medical image capture device from a first viewpoint, and additional information of the scene;
determining, for the medical image capture device, in accordance with the additional information and previous viewpoint information of surgical scenes, one or more candidate viewpoints from which to obtain an image of the surgical scene;
providing, in accordance with the first image of the surgical scene, for each of the one or more candidate viewpoints, a simulated image of the surgical scene from the candidate viewpoint;
controlling the medical image capture device to obtain an image of the surgical scene from the candidate viewpoint corresponding to a selection of one of the one or more simulated images of the surgical scene.

21. A non-transitory computer program product including instructions which, when the program is executed by a computer, cause the computer to carry out a method of controlling a medical image capture device during surgery, the method comprising:
receiving a first image of the surgical scene, captured by the medical image capture device from a first viewpoint, and additional information of the scene;
determining, for the medical image capture device, in accordance with the additional information and previous viewpoint information of surgical scenes, one or more candidate viewpoints from which to obtain an image of the surgical scene;
providing, in accordance with the first image of the surgical scene, for each of the one or more candidate viewpoints, a simulated image of the surgical scene from the candidate viewpoint;
controlling the medical image capture device to obtain an image of the surgical scene from the candidate viewpoint corresponding to a selection of one of the one or more simulated images of the surgical scene.
